(19) United States
(12) Patent Application Publication
(10) Pub. No.: US 2004/0185068 A1
(43) Pub. Date: Sep. 23, 2004

(54) Self-emulsifying compositions, methods of use and preparation

(76) Inventors: Zhi-Jian Yu, Irvine, CA (US); Stan Huth, Newport Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/392,375

(22) Filed: Mar. 18, 2003

(51) Int. Cl. A61K 7/00

(52) U.S. Cl. 424/401

(57) ABSTRACT

Preparation of self-emulsifying compositions which are prepared without mechanical homogenization is described. These self-emulsifying compositions are prepared using one or two surfactants. This provides the advantage of a low weight ratio of emulsifying component to oil component and fewer chemical toxicity concerns, resulting in comfort and safety advantages over emulsions employing more than two emulsifiers. Consequently, the self-emulsifying compositions described are ideally suited for ophthalmic applications including administration of therapeutics to the eye. Self-emulsifying compositions prepared by the disclosed method are described.

### BACKGROUND OF THE INVENTION

1. Field of the Invention

In one embodiment, the present invention relates to nanotechnology and self-emulsifying compositions, including ophthalmic compositions and methods of making and using same. These emulsions employ molecular self-assembly to generate oil droplet structures at the nanometer and sub-micron scale.

2. Description of the Related Art

Typical preparation of oil-in-water emulsions has involved dissolving water-soluble components in an aqueous phase and dissolving oil-soluble components in an oil phase. The oil phase is vigorously dispersion mixed into the aqueous phase at several thousand r.p.m. for minutes to several hours. Manufacturing procedures employing such methods involve significant investment in capital equipment, are time consuming and cannot be easily scaled-up to larger batch sizes. Also, it is generally difficult to stabilize oil-in-water emulsions prepared by these types of methodologies for a commercially desired shelf-life of two years without incorporating viscosity builders. However, high viscosity is often undesirable for ophthalmic solutions and almost universally unacceptable for contact lens care solutions. A two-year shelf life can sometimes be achieved if the emulsions are stored refrigerated. However, the use of refrigeration limits commercial distribution of the product.

Sterilization is essential for many oil-in-water emulsions, which readily support the growth of bacteria, the latter which give rise to contamination of the composition. A problem encountered with emulsions prepared by standard methods is that they are not easily sterilized using filtration techniques. Filter sterilization for ophthalmic compositions which comprise oil-in-water emulsions is preferred to heat sterilization because of problems associated with heat sterilization such as manufacturing complexity and cost. Also, precipitation and/or inactivation of composition components may occur in sterilization procedures where heat is used.

Additionally, oil-in-water emulsions prepared via conventional methods generally require high surfactant to oil ratios. Oil-in-water emulsions with a low surfactant to oil ratio generally produce a higher degree of ocular comfort than those with a high surfactant to oil ratio. Ocular comfort is of critical importance for commercial success in products such as eye drops and contact lens multipurpose solutions.

Additionally, oil-in-water emulsions prepared via conventional methods generally require two or more surfactants, resulting in high surfactant to oil ratios. Such oil-in-water emulsions are described in U.S. application Ser. No. 10/349,466, filed Jan. 22, 2003, which is incorporated herein by reference. This leads to problems with achieving low toxicity as increasing complexity of the compositions.

In view of these and other limitations to oil-in-water emulsions prepared by standard techniques, it would be advantageous to have oil-in-water emulsions which are easily prepared and sterilized and which are storage stable. It is an object of this invention to provide such compositions as well as methods of preparing such compositions. These ophthalmic compositions positions have a low surfactant to oil ratio for applications requiring high comfort, and employ fewer surfactants to achieve emulsification. These compositions employ molecular self-assembly methods to generate macromolecular oil droplet structures at the nanometer scale, and thus represent an example of nanotechnology.

### SUMMARY OF THE INVENTION

Self-emulsifying oil-in-water emulsion compositions, methods of use and preparation are described. In a preferred embodiment, self-emulsifying ophthalmic compositions, methods of use and preparation are described. In one embodiment, a self-emulsifying composition is described which includes oil globules having an average size of less than 1 micron dispersed in an aqueous phase. The globules contain a surfactant component containing one or two surfactants; and a polar oil component. The surfactant component and the oil component are selected to self-emulsify when mixed without mechanical homogenization. It is noted that the surfactant component may contain other surfactants that do not contribute to the self-emulsification.

In one embodiment, the surfactant component of the self-emulsifying compositions described herein has a hydrophobic portion which includes a first part oriented proximal to the aqueous phase that is larger than a second part of the hydrophobic portion of the surfactant component oriented towards the interior of the oil globule. In a preferred embodiment, the surfactant component contains one surfactant and the first part of the hydrophobic portion of the surfactant contains more atoms than the second part of the hydrophobic portion of the surfactant.

In an alternate preferred embodiment, the surfactant component contains two surfactants. A first surfactant has a first hydrophobic portion and a second surfactant has a second hydrophobic portion. The first hydrophobic portion of the first surfactant has a longer chain length than the second hydrophobic portion of the second surfactant.

In some embodiments, the self-emulsifying composition may include an additional surfactant(s) that does not interfere with self-emulsification.

In preferred embodiments, the oil component of the self-emulsifying composition may include castor oil or other natural oils.

In preferred embodiments, the surfactant component is selected from compounds having at least one ether formed from at least about 1 to 100 ethylene oxide units and at least one fatty alcohol chain having from at least about 12 to 22 carbon atoms; compounds having at least one ester formed from at least about 1 to 100 ethylene oxide units and at least one fatty acid chain having from at least about 12 to 22 carbon atoms; compounds having at least one ether, ester or amide formed from at least about 1 to 100 ethylene oxide units and at least one vitamin or vitamin derivative; and combinations thereof consisting of no more than two surfactants.

In a particularly preferred embodiment, the surfactant component is Lumulse GRH-40. In an alternate preferred embodiment, the surfactant component is TGPS.

Preferably, the oil globules have an average size of less than 0.25 micron and more preferably, less that 0.15 micron.

The self-emulsifying compositions may be used in a therapeutic composition which includes the self-emulsifying compositions described herein in combination with a therapeutic drug. In a preferred embodiment, the therapeutic drug may be cyclosporin, prostaglandins, Brimonidine, or Brimonidine salts. In a preferred embodiment, the oil is a natural oil such as castor oil. In a most preferred embodiment, the therapeutic compositions contain a single surfactant which is Lumulse GRH-40.

Ophthalmic compositions containing the self-emulsifying compositions described herein are particularly preferred and include the self-emulsifying composition described above in combination with a drug that is therapeutic when administered to the eye. In a preferred embodiment, the oil is a natural oil such as castor oil. In a most preferred embodiment, the ophthalmic compositions contain a single surfactant which is Lumulse GRH-40.

Another aspect of the invention is directed to methods of preparing the self-emulsifying composition described herein which includes the steps of:

preparing an oil phase which includes a polar oil and a surfactant component that contains one or two surfactants, where the polar oil and the surfactant component in the oil phase are in the liquid state;

preparing an aqueous phase at a temperature that permits self-emulsification; and

mixing the oil phase and the aqueous phase to form an emulsion, without mechanical homogenization.

In a preferred embodiment, the method includes the step of forming a paste between the oil phase and a part of the aqueous phase and mixing the paste with the rest of the aqueous phase to form an emulsion.

In one embodiment, self-emulsifying compositions are described which are capable of being produced by the steps of first preparing an oil phase which includes a polar oil and a surfactant component that contains one or two surfactants, wherein the polar oil and the surfactant component in the oil phase are in the liquid state. Second, preparing an aqueous phase at a temperature that permits self-emulsification. Finally, mixing the oil phase and the aqueous phase to form an emulsion, without mechanical homogenization.

In one embodiment, the self-emulsifying compositions produced by methods described herein include a surfactant component which is a single surfactant. In preferred embodiments, the oil is a natural oil, preferably castor oil. In preferred embodiments, the surfactant component may be a compound having at least one ether formed from at least about 1 to 100 ethylene oxide units and at least one fatty alcohol chain having from at least about 12 to 22 carbon atoms; a compound having at least one ester formed from at least about 1 to 100 ethylene oxide units and at least one fatty acid chain having from at least about 12 to 22 carbon atoms; a compound having at least one ether, ester or amide formed from at least about 1 to 100 ethylene oxide units and at least one vitamin or vitamin derivative; and combinations thereof consisting of no more than two surfactants.

In a most preferred embodiment, the surfactant component is Lumulse GRH-40. In an alternate preferred embodiment, the surfactant component is TGPS.

The present invention also includes therapeutic compositions containing self-emulsifying compositions prepared by the methods described herein in combination with a therapeutic drug. In preferred embodiments, the therapeutic compounds are selected from cyclosporin, prostaglandins, Brimonidine, and Brimonidine salts. In a preferred embodiment, the oil is a natural oil such as castor oil. In a most preferred embodiment, the therapeutic compositions contain a single surfactant which is Lumulse GRH-40.

The present invention also includes ophthalmic compositions containing the self-emulsifying compositions prepared by methods described herein in combination with a drug that is therapeutic when administered to the eye. In a preferred embodiment, the oil is a natural oil such as castor oil. In a most preferred embodiment, the ophthalmic compositions contain a single surfactant which is Lumulse GRH-40.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1 shows a flow chart for the preparation of the ophthalmic self-emulsifying compositions described.

FIG. 2 shows results of cytotoxicity studies for sample formulations. BAK 200 ppm (-∘-), 29BB (-▴-), 30U (-*-), 83A (-□-), 51C (-Δ-), 82B (-□-), Endura (-∘-), 34AA (-&Rectsolid;-), 35A (-⋄-).

FIG. 3 shows results of cytotoxicity studies for sample formulations. BAK 200 ppm (-●-), 44A (-▴-), 48B (-*-), 47A (-▪-), 98C (-Δ-), 52A (-□-), Endura (-∘-), 83A (-&Rectsolid;-), 53B (-⋄-).

FIG. 4 shows results of cytotoxicity studies for sample formulations. BAK 200 ppm (-●-), 57A (-▴-), 57D (-*-), 58B (-▪-), 58E (-Δ-), 59C (-□-), 59F (-∘-), 60A (-&Rectsolid;-), 59G (-⋄-).

FIG. 5 shows results of cytotoxicity studies for sample formulations. BAK 200 ppm (-●-), 76A (-▴-), 76B (-*-), 76C (-▪-), 76D (-Δ-), 75A (-□-), Endura (-∘-).

FIG. 6 shows results of cytotoxicity studies for sample formulations. BAK 200 ppm (-●-), 75A (-▴-), 75B (-*-), 75C (-▪-), 73D (-{-), 73E (-□-), Endura (-∘-).

FIG. 7 shows results of cytotoxicity studies for sample formulations. BAK 200 ppm (-●-), 73F (-▴-), 73G (-*-), 73H (-▪-), 73I (-Δ-), 75A (-□-), Endura (-∘-).

### DETAILED DESCRIPTION OF THE PREFERRED EMBODIMENT

Novel enhanced ophthalmic compositions comprising oil-in-water emulsions, preferably self-emulsifying oil-in-water emulsions, methods of preparing or making such compositions and methods of using such compositions have been discovered with unexpectedly improved results within the field. The present emulsion-containing compositions are relatively easily and straight forwardly prepared and are storage-stable, for example, having a shelf life at about room temperature of at least about one year or about 2 years or more. In addition, the present compositions are advantageously easily sterilized, for example, using sterilizing filtration techniques, and eliminate, or at least substantially reduce, the opportunity or risk for microbial growth if the compositions become contaminated.

The present compositions preferably include self-emulsifying emulsions. That is, the present oil-in-water emulsions preferably can be formed with reduced amounts of dispersion mixing at shear speed, more preferably with substantially no dispersion mixing at shear speed. Dispersion mixing at shear speed is also known as mechanical homogenization. Mechanical homogenization to form an emulsion typically occurs at shear speeds greater than 1000 r.p.m., more typically at several thousand r.p.m., and even at 10,000 r.p.m. or more. In other words, the present self-emulsifying emulsions preferably can be formed using reduced amounts of shear, and more preferably using substantially no shear. Further, the present emulsions have a relatively low weight ratio of emulsifying component or surfactant component to oil or oily component and, therefore, are advantageously safe and comfortable for topical ophthalmic application. Such oil-in-water emulsions, with a low surfactant to oil ratio, may be more readily prepared via self-emulsification than oil-in-water emulsions with a higher surfactant to oil ratio.

Topical ophthalmic application forms of the present compositions include, without limitation, eye drops for dry eye treatment and for other treatments, forms for the delivery of drugs or therapeutic components into the eye and forms for caring for contact lenses. The present compositions are very useful for treating dry eye and similar conditions, and other eye conditions. In addition, the present compositions are useful in or as carriers or vehicles for drug delivery, for example, a carrier or vehicle for delivery of therapeutic components into or through the eyes.

Contact lens care applications of the present compositions include, without limitation, compositions useful for cleaning, rinsing, disinfecting, storing, soaking, lubricating, re-wetting and otherwise treating contact lenses, including compositions which are effective in performing more than one of such functions, i.e., so called multi-purpose contact lens care compositions, other contact lens care-related compositions and the like. Contact lens care compositions including the present emulsions also include compositions which are administered to the eyes of contact lens wearers, for example, before, during and/or after the wearing of contact lenses.

The integration of emulsions into contact lens care compositions, such as multi-purpose, re-wetting and other contact lens care compositions adds the additional utility or benefit of prevention of dry eye and provides lubrication to the lens and/or eye through mechanisms only emulsions can provide. Additional utilities or benefits provided by integrated emulsions in contact lens care compositions may include, without limitation, enhanced contact lens cleaning, prevention of contact lens water loss, inhibition of protein deposition on contact lenses and the like.

The present invention provides for ophthalmic compositions which include oil-in-water emulsions, preferably self-emulsifying oil-in-water emulsions. These oil-in-water emulsions comprise an oil component, for example, and without limitation, castor oil; and an aqueous component which includes two emulsifiers or surfactants or less. The use of only one or two emulsifiers results in a low weight ratio of emulsifying component to oil component and fewer chemical toxicity concerns, resulting in comfort and safety advantages over emulsions employing more than two emulsifiers.

The oily component and the surfactant component or surfactants are advantageously chemically structurally compatible to facilitate self-emulsification of the emulsion. In the context of the present invention, surfactant component means one or two surfactants that are present in the self-emulsifying composition and contribute to the self-emulsification. The one or two surfactants must have an affinity for the selected oil or oils based upon non-covalent bonding interactions between the hydrophobic structures of the surfactant and the oil(s) such that self emulsification can be achieved. In one aspect, affinity relates to the use of a polar oil with a surfactant of similar polarity. As the terms are used herein, a polar oil means that the oil contains heteroatoms such as oxygen, nitrogen and sulfur in the hydrophobic part of the molecule. In a preferred embodiment, the self-emulsifying emulsions described contain at least one polar oil.

Additionally, the one or two surfactants must be able to form a chemical structure which is wedge or pie section-shaped, with the larger end of the wedge structure closer to the hydrophilic parts of the surfactant structures. That is, the part of the surfactant that is larger is oriented towards the aqueous phase and contains more atoms than the part of the surfactant that is oriented towards the oil phase. When the surfactant component includes two surfactants, the hydrophobic portion of the first surfactant may have a longer chain length than the hydrophobic portion of the second surfactant to promote formation of a wedge shape.

The surfactants useful to form the surfactant component in the present invention advantageously are water-soluble when used alone or as a mixture. These surfactants are preferably non-ionic. The amount of surfactant component present varies over a wide range depending on a number of factors, for example, the other components in the composition and the like. Often the total amount of surfactant component is in the range of about 0.01 to about 10.0 w/w %. It is noted that additional surfactant(s) may be present in the self-emulsifying composition and still fall within the scope of the present invention if the additional surfactant(s) are present at a concentration such that they do not interfere with the self-emulsification.

The ratio, for example, weight ratio, of the surfactant component to the oily component in the present oil-in-water emulsions is selected to provide acceptable emulsion stability and performance, and preferably to provide a self-emulsifying oil-in-water emulsion. Of course, the ratio of surfactant component to oily component varies depending on the specific surfactants and oil or oils employed, on the specific stability and performance properties desired for the final oil-in-water emulsion, on the specific application or use of the final oil-in-water emulsion and the like factors. For example, the weight ratio of the surfactant component to the oily component may range from about 0.05 to about 20.

Such surfactants function as described herein, provide effective and useful ophthalmic compositions and do not have any substantial or significant detrimental effect on the contact lens being treated by the present compositions, on the wearers of such contact lenses or on the humans or animals to whom such compositions are administered.

The ophthalmic compositions comprise an oily component which may include, without limitation, castor oil and the like. One or more oils or oily substances are used to form the present compositions. Any suitable oil or oily substance or combinations of oils or oily substances may be employed provided such oils and/or oily substances are effective in the present compositions, and do not cause any substantial or significant detrimental effect to the human or animal to whom the composition is administered, or to the contact lens being treated, or the wearing of the treated contact lens, or to the wearer of the treated contact lens. The oily component may, for example, and without limitation, be polar in nature and naturally or synthetic derived. Natural oils may be obtained from plants or plant parts such as seeds or they may be obtained from an animal source such as Sperm Whale oil, Cod liver oil and the like. The oil may be a mono, di or triglyceride of fatty acids or mixtures of glycerides, such as Castor oil, Coconut oil, Cod-liver oil, Corn oil, Olive oil, Peanut oil, Safflower oil, Soybean oil and Sunflower oil. The oil may also be comprised of straight chain monoethylene acids and alcohols in the form of esters, such as Jojoba and Sperm Whale oil. The oil may be synthetic, such as silicone oil. The oil also may be comprised of water insoluble non-volatile liquid organic compounds, e.g., a racemic mixture of Vitamin E acetate isomers. Mixtures of the above oil types may also be used.

Oils which are natural, safe, have prior ophthalmic or other pharmaceutical use, have little color, do not easily discolor upon aging, easily form spread films and lubricate surfaces without tackiness are preferred. Castor oil is a preferred oil.

In one embodiment, the present invention relates to ophthalmic compositions which are self-emulsifying, oil-in-water emulsions as well as methods of preparing and methods of using such ophthalmic compositions. These compositions are useful for eye and contact lens care. These emulsions employ molecular self-assembly methods to generate macromolecular oil droplet structures at the nanometer and sub-micron scale and thus represent an example of nanotechnology. The emulsions are easily prepared via molecular self-assembly in milliseconds to minutes. The emulsions can be filter sterilized and are storage-stable. The emulsions employ only one or two surfactant emulsifiers to achieve low surfactant to oil ratios. The compositions are comfortable and non-toxic to the eye.

Topical ophthalmic applications for the emulsions of the present invention include eye drops for dry eye treatment, compositions for delivery of drugs to and via the eye, and contact lens care solutions. Contact lens care solution applications include multipurpose cleaning, rinsing, disinfecting and storage solutions as well as rewetting, in-the-eye cleaning and other solutions for the eye.

The integration of oil-in-water emulsions into eye drops for dry eye treatment, contact lens rewetting and multipurpose solutions adds the additional utility of prevention of dry eye and contact lens water loss by providing an oil layer at the air-tear interface or additionally at the contact lens-tear interface when a contact lens is present. This oil layer acts to prevent dry eye or contact lens water loss by retarding water evaporation and thus loss. The oil layer on the surface of a contact lens can also provide a long-lasting lubrication layer, especially for rigid gas permeable contact lenses. The oil layer on the surface of a contact lens can also inhibit contact lens protein deposition.

The self-emulsifying, oil-in-water emulsions of the present invention are of two general types. The first type is a one surfactant system. The second type is a two surfactant system. In either case, what is required is that (1) the surfactant(s) must have an affinity for the selected oil or oils based upon non-covalent bonding interactions between the hydrophobic structures of the surfactant and the oil(s) such that self emulsification can be achieved when requirement (2) is simultaneously met; and (2) the surfactant must have a chemical structure which is wedge or pie section-shaped, with the larger end of the wedge structure closer to the hydrophilic part of the surfactant structure. This wedge-shape is believed to induce spherical oil droplet curvature at the aqueous-oil interface due to the molecular self-assembly of adjacent surfactant wedges at the aqueous-oil interface. Thus, the geometry of the wedge-shaped surfactant molecules is intimately related to the oil droplet curvature. Steric repulsion in the aqueous phase between the hydrophilic parts of adjacent surfactant molecules facilitates this. Preferably, these hydrophilic parts consist of polyethyleneoxide chains of an appropriate length. Preferably, the polyethyleneoxide chains are from 7-20 ethyleneoxide units in length. When the aforementioned two structural requirements are met for a surfactant and oil(s) pair(s), an empirical test of self emulsification is conducted while varying the concentrations of the surfactant and oil components. The empirical test of self emulsification is conducted employing the methods of preparing self emulsifying emulsions described herein. An emulsion is considered to be acceptable when it appears to be homogeneous when observed by eye, without any appearance of flocculation, cream or phase separation between the aqueous and oil phase and also when the oil droplet size distribution of the emulsion meets particular product criteria for emulsion stability.

As a practical matter, a surfactant is a good candidate for the self-emulsifying oil-in-water emulsions described herein if the surfactant is able to form droplets of a size range of 0.05 to 1 micron, preferably, 0.05 to 0.25 micron.

Examples of one component surfactant systems include polyethoxylated oils such as PEG castor oils. Polyethoxylated castor oil derivatives are formed by the ethoxylation of castor oil or hydrogenated castor oil with ethylene oxide. Castor oil is generally composed of about 87% ricinoleic acid, 7% oleic acid, 3% linoleic acid, 2% palmitic acid and 1% stearic acid. The reaction of varying molar ratios of ethylene oxide with castor oil yields different chemical products of PEG castor oils. An example of a PEG castor oil is Lumulse GRH-40, produced by Lambent Technologies Corporation (Skokie, Ill.). A preferred example of a single surfactant and oil pair is the surfactant Lumulse GRH-40 and Castor oil.

Lumulse GRH-40 is a 40 mole ethoxylate of hydrogenated Castor oil. Lumulse GRH-40 is produced through the catalytic hydrogenation of Castor oil at the 9-carbon positions of the three ricinoleic acid glycerol ester chains, followed by ethoxylation of the three 12-hydroxy groups of the 12-hydroxystearic acid glycerol esters with about 13 ethoxy groups each. It is believed that self emulsification of Castor oil with Lumulse GRH-40 occurs due to the folding of the 6-carbon alkyl chain distal to the ethoxylated 12-hydroxy group inwards against the remaining 10-carbon alkyl segment of the stearate ester group to form a wedge-shaped hydrophobic part of the molecule, the orientation of the ethoxy groups outwards into the water phase, the orientation of the wedge-shaped hydrophobic part of the molecule into the Castor oil phase (narrow part of the wedge facing inwards away from the aqueous phase) and the affinity of the wedge-shaped hydrophobic part of the molecule for Castor oil.

The optimal amount of Lumulse GRH-40 to use in conjunction with Castor oil is about 0.8 w/w % Lumulse GRH-40 for 1.0 w/w % Castor oil. Higher or lower amounts in conjunction with Castor oil can be used, however, depending upon the desired properties of the final emulsion.

Lumulse GRH-40 can be combined with other surfactants such as Polysorbate-80 (Tween-80, polyoxyethylene (20) sorbitan mono-oleate) to create self-emulsifying emulsions comprised of two surfactants. In such compositions, self emulsification is believed to be driven principally by the Lumulse GRH-40. The second surfactant (e.g. polysorbate-80) does not interfere with the emulsifying action of the GRH-40 due to the similar chemical structures of the hydrophobic chains of Polysorbate-80 (oleic acid ester chains) and those of Castor oil (12-hydroxyoleic acid ester chains) and Lumulse GRH-40 (stearic acid ester chains). The non-interfering second surfactant is present at low concentration. That is, the concentration of the non-interfering surfactant is low enough such that it does not interfere with the self-emulsification.

Two surfactants may also be selected to match a particular oil or oils with respect to the ability of the surfactants to form a self-emulsifying oil-in-water emulsion. Both surfactants must each meet two chemical structural requirements to achieve self emulsification: (1) each surfactant must have an affinity for the selected oil or oils based upon non-covalent bonding interactions between the hydrophobic structures of the surfactant and the oil(s) such that self emulsification can be achieved when requirement (2) is simultaneously met; and (2) the surfactant pair must be able to form a chemical structure which is wedge or pie section-shaped, with the larger end of the wedge structure closer to the hydrophilic parts of the surfactant structures. A preferred example of a surfactant pair which is compatible with an oil is the surfactant raw material Cremophor RH-40, which is comprised of two surfactants, and Castor oil. Cremophor RH-40, from the BASF Corporation in Parsippany N.J., is comprised 75-78% of two surfactants: the trihydroxystearate ester of polyethoxylated glycerol and the hydroxystearate (bis) ester of mixed polyethylene glycols, along with 22-25% free polyethylene glycols. The Cremophor RH-40 raw material thus has two surfactants which are structurally related to each other and to Castor oil. It is believed that the combination of a surfactant with three ester chains with a surfactant with two ester chains, wherein all of the chains have an affinity for each other, allows the formation of a wedge-shaped structure in the presence of Castor oil wherein the two surfactants alternate at the oil droplet interface. Cremophor RH-60, also from BASF, is an example of another surfactant raw material comprised of two surfactants. Cremophor RH-60 is identical to Cremophor RH-40, with the exception that there is a higher derivatization with polyethyleneglycol with RH-60 than with RH-40.

Additional surfactant may be added which may or may not participate in emulsion formation.

Another example of a one component system utilizes a surfactant such as tocopherol polyethyleneglycol-succinate (TPGS, available from Eastman Chemical Company, Kingsport, Tenn.). TPGS can form a wedge with tocopherol in the narrow section, PEG in the outer section and succinate forming a covalent attachment between them.

More generic descriptions of the types of surfactants which can be used in the present invention include surfactants selected from the group comprising: (a) at least one ether formed from 1 to 100 ethylene oxide units and at least one fatty alcohol chain having from 12 to 22 carbon atoms; (b) at least one ester formed from 1 to 100 ethylene oxide units and at least one fatty acid chain having from 12 to 22 carbon atoms; (c) at least one ether, ester or amide formed from 1 to 100 ethylene oxide units and at least one vitamin or vitamin derivative, and (d) mixtures of the above consisting of no more than two surfactants.

The preparation of the oil-in-water emulsions of the present invention is generally as follows. Non-emulsifying agents which are water soluble components are dissolved in the aqueous (water) phase and oil-soluble components including the emulsifying agents are dissolved in the oil phase. The two phases (oil and water) are separately heated to an appropriate temperature. This temperature is the same in both cases, generally a few degrees to 5 to 10 degrees above the melting point of the highest melting ingredients in the case of a solid or semi-solid oil or emulsifying agent in the oil phase. Where the oil phase is liquid at room temperature, a suitable temperature is determined by routine experimentation with the melting point of the highest melting ingredients in the aqueous phase. In cases where all components of either the oil or water phase are soluble in their respective phase at room temperature, no heating may be necessary. The temperature must be high enough that all components are in the liquid state but not so high as to jeopardize the stability of the components. A working temperature range is generally from about 20° C. to about 70° C. To create an oil-in-water emulsion, the final oil phase is gently mixed into either an intermediate, preferably de-ionized water phase, or the final aqueous phase to create a suitable dispersion and the product is allowed to cool with or without stirring. In the case wherein the final oil phase is first gently mixed into an intermediate water phase, this emulsion concentrate is thereafter mixed in the appropriate ratio with the final aqueous phase. In such cases, the emulsion concentrate and the final aqueous phase need not be at the same temperature or heated above room temperature, as the emulsion has already been formed at this point.

Semisolids may form in the process of self-emulsification if the amount of ethylene oxide units in one emulsifier is too large. Generally, if the surfactant or surfactants have more than 10 ethylene oxide units in their structures, the surfactant and oil phase is mixed with a small amount of the total composition water, e.g., about 0.1-10%, to first form a paste, which is thereafter combined with the remaining water. Gentle mixing may then be required until the hydrated emulsifiers are fully dissolved to form the emulsion.

In one embodiment, the surfactant and oil are initially combined and heated. A small amount of the aqueous phase is then added to the oil phase to form a paste. Paste is defined here as a semisolid preparation which does not flow. The amount of the aqueous phase added may be from 0.1-10%, preferably from 0.5 to 5% and most preferably 1-2%. After the paste is formed, additional water is added to the paste at the same temperature as above. In some embodiments, the amount of water added is 5-20%. The emulsion is then gently mixed. In some embodiments, mixing may occur for 30 minutes to 3 hours.

In a preferred embodiment, the particles are then sized. A Horiba LA-920 particle size analyzer may be used according to the manufacturer's instructions for this purpose. In a preferred embodiment, the particles are between 0.08 and 0.18 microns in size before passing to the next step.

In the next step, the particles may be mixed with other aqueous components such as water and buffer (preferably boric acid based). Optionally, electrolytes, such as calcium chloride dihydrate, magnesium chloride hexahydrate, potassium chloride and sodium chloride, and Kollidon 17 NF may be added. While the electrolytes are not necessary to form the emulsions, they are very helpful to preserve ocular tissue integrity by maintaining the electrolyte balance in the eye. Likewise, the buffer is not critical, but a boric acid/sodium borate system is preferred because a phosphate-based buffer system will precipitate with the preferred electrolytes.

The pH is adjusted to 6.8-8.0, preferably from about 7.3 to 7.7. This pH range is optimal for tissue maintenance and to avoid ocular irritation. A preservative may then be added. In a preferred embodiment, Purogene® material is added as preservative. (PUROGENE is a trademark of BioCide International, Inc. Norman, Okla., U.S.A., and is also available as Purite® which is a trademark of Allergan, Inc.)

The oil-in-water emulsions of the present invention can be sterilized after preparation using autoclave steam sterilization or can be sterile filtered by any means known in the art such as by using a 0.22 micron sterile filter. Sterilization employing a sterilization filter can be used when the emulsion droplet (or globule or particle) size and characteristics allows. The droplet size distribution of the emulsion need not be entirely below the particle size cutoff of the sterile filtration membrane to be sterile-filtratable. In cases where the droplet size distribution of the emulsion is above the particle size cutoff of the sterile filtration membrane, the emulsion needs to be able to deform or acceptably change while passing through the filtrating membrane and then reform after passing through. This property is easily determined by routine testing of emulsion droplet size distributions and percent of total oil in the compositions before and after filtration. Alternatively, a loss of a small amount of larger droplet-sized material may be acceptable.

The emulsions of the present invention are generally non-aseptically filtered through a clarification filter before sterile filtration or aseptically clarify-filtered after autoclave steam sterilization. In a preferred embodiment, the emulsion is filter sterilized using a 0.22 micron filter. Preferably, 98-99% of the emulsion should pass through the 0.22 micron filter. Note that particles larger than 0.22 micron may pass through by altering their shape temporarily. In a preferred embodiment, the material is then tested to verify the effectiveness of the sterilization step. Storage is preferably below 25° C. in order to maintain stability. Thereafter, the emulsions are aseptically filled into appropriate containers.

The present invention provides for methods of using ophthalmic compositions, such as the present ophthalmic compositions described elsewhere herein. In one embodiment, the present methods comprise administering a composition of the invention to an eye of a subject, for example, a human or an animal, in an amount and at conditions effective to provide at least one benefit to the eye. In this embodiment, the present composition can employ at least one portion of the composition, for example, a therapeutic component and the like, useful for treating a condition, for example, dry eye and/or one or more other conditions of the eye.

In a very useful embodiment, the present methods comprise contacting a contact lens with a composition of the present invention in an amount and at conditions effective to provide at least one benefit to the contact lens and/or the wearer of the contact lens. In this embodiment, the present composition is employed as at least a portion of a contact lens care composition.

When the present compositions include a therapeutic component, such compositions may be used in methods which comprise administering the composition to an eye of a subject, that is a human or animal, in an amount effective in providing a desired therapeutic effect to the subject. Such therapeutic effect may be an ophthalmic therapeutic effect and/or a therapeutic effect directed to one or more other parts of the subject's body or systemically to the subject's body. In this embodiment, the present oil-in-water emulsion is employed as at least a portion of a composition useful as a carrier or vehicle for the therapeutic component.

The aqueous phase or component and the oil phase and component used in accordance with the present invention are selected to be effective in the present compositions and to have no substantial or significant deleterious effect, for example, on the compositions, on the use of the compositions, on the contact lens being treated, on the wearer of the treated lens, or on the human or animal in whose eye the present composition is placed.

The liquid aqueous medium or component of the present compositions preferably includes a buffer component which is present in an amount effective to maintain the pH of the medium or aqueous component in the desired range. The present compositions preferably include an effective amount of a tonicity adjusting component to provide the compositions with the desired tonicity.

The aqueous phase or component in the present compositions may have a pH which is compatible with the intended use, and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Phosphate buffers include phosphoric acid and its salts; for example, M₂HPO₄ and MH₂PO₄, wherein M is an alkali metal such as sodium and potassium. Hydrated salts can also be used. In one embodiment of the present invention, Na₂HPO₄. 7H₂O and NaH₂PO₄.H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v% and more preferably varies from about 0.05 to about 0.5 w/v %.

The type and amount of buffer are selected so that the formulation meets the functional performance criteria of the composition, such as surfactant and shelf life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of about 7.45, is very useful, although a wider pH range from about 6 to about 9, more preferably about 6.5 to about 8.5 and still more preferably about 6.8 to about 8.0 is also acceptable. In one embodiment, the present composition has a pH of about 7.0.

The osmolality of the present compositions may be adjusted with tonicity agents to a value which is compatible with the intended use of the compositions. For example, the osmolality of the composition may be adjusted to approximate the osmotic pressure of normal tear fluid, which is equivalent to about 0.9 w/v% of sodium chloride in water. Examples of suitable tonicity adjusting agents include, without limitation, sodium, potassium, calcium and magnesium chloride; dextrose; glycerin; propylene glycol; mannitol; sorbitol and the like and mixtures thereof. In one embodiment, a combination of sodium chloride and potassium chloride are used to adjust the tonicity of the composition.

Tonicity agents are typically used in amounts ranging from about 0.001 to 2.5 w/v%. These amounts have been found to be useful in providing sufficient tonicity for maintaining ocular tissue integrity. Preferably, the tonicity agent(s) will be employed in an amount to provide a final osmotic valve of 150 to 450 mOsm/kg, more preferably between about 250 to about 330 mOsm/kg and most preferably between about 270 to about 310 mOsm/kg. The aqueous component of the present compositions more preferably is substantially isotonic or hypotnoic (for example, slightly hypotonic, e.g., about 240 mOsm/kg) and/or is ophthalmically acceptable. In one embodiment, the compositions contain about 0.14 w/v% potassium chloride and 0.006 w/v% each of calcium and/or magnesium chloride.

In addition to tonicity and buffer components, the present compositions may include one or more other materials, for example, as described elsewhere herein, in amounts effective for the desired purpose, for example, to treat contact lenses and/or ocular tissues, for example, to provide a beneficial property or properties to contact lenses and/or ocular tissues, contacted with such compositions.

In one embodiment, the compositions of the present invention are useful, for example, as a carrier or vehicle, for the delivery of therapeutic agents to or through the eye. Any suitable therapeutic component may be included in the present compositions provided that such therapeutic component is compatible with the remainder of the composition, does not unduly interfere with the functioning and properties of the remainder of the composition, is effective, for example, to provide a desired therapeutic effect, when delivered in the present composition and is effective when administered to or through the eye. For example, in a very useful embodiment, the delivery of hydrophobic therapeutic components or drugs to or through the eye may be accomplished. Without wishing to limit the invention to any particular theory or mechanism of operation, it is believed that the oily component and the hydrophobic constituents of the surfactant components facilitate hydrophobic therapeutic components remaining soluble, stable and effective in the present compositions.

According to this aspect of the invention, an effective amount of a desired therapeutic agent or component preferably is physically combined or mixed with the other components of a composition of the present invention to form a therapeutic component-containing composition within the scope of the present invention.

While compositions for the delivery of therapeutic agents to or through the eye are a preferred embodiment, the self-emulsifying compositions described herein can be use for delivery of therapeutics through other means including, but not limited to oral, rectal, vaginal, parenteral, intramuscular, intraperitoneal, intraarterial, intrathecal, intrabronchial, subcutaneous, intradermal intravenous, nasal, buccal and sublingual.

The type of therapeutic agent or agents used will depend primarily on the therapeutic effect desired, for example, the disease or disorder or condition to be treated. These therapeutic agents or components include a broad array of drugs or substances currently, or prospectively, delivered to or through the eye in topical fashion or otherwise. Examples of useful therapeutic components include, but not limited to:

(1) anti-infective and anti-microbial substances including quinolones, such as ofloxacin, ciprofloxacin, norfloxacin, gatifloxacin and the like; beta-lactam antibiotics, such as cefoxitin, n-formamidoyl-thienamycin, other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin sulfonamides and the like; aminoglycoside antibiotics, such as gentamycin, kanamycin, amikacin, sisomicin, tobramycin and the like; naladixic acid and analogs thereof and the like; antimicrobial combinations, such as fluealanine/pentizidone and the like; nitrofurazones; and the like and mixtures thereof;

(2) anti-allergy agents, antihistaminics, anti-hypertensive agents and decongestants, such as pyrilamine, chlorpheniramine, phenylephrine hydrochloride, tetrahydrazoline hydrochloride, naphazoline hydrochloride, oxymetazoline, antazoline, and the like and mixtures thereof;

(3) anti-inflammatories, such as cortisone, hydrocortisone, hydrocortisone acetate, betamethansone, dexamethasone, dexamethasone sodium phosphate, prednisone, methylprednisolone, medrysone, fluorometholone, fluocortolone, prednisolone, prednisolone sodium phosphate, triamcinolone, sulindac, salts and corresponding sulfides thereof, and the like and mixtures thereof;

(4) non-steroid anti-inflammatory drug (NSAD) components, such as those which do or do not include a carboxylic (—COOH) group or moiety, or a carboxylic derived group or moiety; NSAID components which inhibit, either selectively or non-selectively, the cyclo-oxygenase enzyme, which has two (2) isoforms, referred to as COX-1 and COX-2; phenylalkoanoic acids, such as diclofenac, flurbiprofen, ketorolac, piroximcam, suprofen and the like; indoles such as indomethacin and the like; diarylpyrazoles, such as celecoxib and the like; pyrrolo pyrroles; and other agents that inhibit prostaglandiin synthesis and the like and mixtures thereof;

(5) miotics and anticholinergics, such as echothiophate, pilocarpine, physostigmine salicylate, diisopropylfluorophosphate, epinephrine, dipivolyl epinephrine, neostigmine, echothiopate iodide, demecarium bromide, carbachol, methacholine, bethanechol, and the like and mixtures thereof;

(6) mydriatics, such as atropine, homatropine, scopolamine, hydroxyamphetamine, ephedrine, cocaine, tropicamide, phenylephrine, cyclopentolate, oxyphenonium, eucatropine, and the like and mixtures thereof;

(7) antiglaucoma drugs, for example, prostaglandins, such as those described in U.S. Pat. Nos. 6,395,787 and 6,294,563, which are herein incorporated by reference in their entirety, adrenergic agonists such as quinoxalines and quinoxaline derivatives, such as (2-imidozolin-2-ylamino) quinoxaline, 5-halide-6-(2-imidozolin-2-ylamino) quinoxaline, for example, 5-bromo-6-(2-imidozolin-2-ylamino) quinoxaline and brimonidine and its derivatives, such as those described in U.S. Pat. No. 6,294,563, which is herein incorporated by reference in its entirety and the like, timolol, especially as the maleate salt and R-timolol and timolol derivatives and a combination of timolol or R-timolol with pilocarpine and the like; epinephrine and epinephrine complex or prodrugs such as the bitartrate, borate, hydrochloride and dipivefrin derivatives and the like; hyperosmotic and dipivefrin derivatives and the like; betaxolol, hyperosmotic agents, such as glycerol, mannitol and urea and the like and mixtures thereof;

(8) antiparasitic compounds and/or anti-protozoal compounds, such as ivermectin; pyrimethamine, trisulfapyrimidine, clindamycin and corticosteroid preparations and the like and mixtures thereof;

(9) antiviral compounds, such as acyclovir, 5-iodo-2′-deoxyuridine (IDU), adenosine arabinoside (Ara-A), trifluorothymidine, interferon and interferon inducing agents, such as Poly I:C and the like and mixtures thereof;

(10) carbonic anhydrase inhibitors, such as acetazolamide, dichlorphenamide, 2-(p-hydroxyphenyl) thio-5-thiophenesulfonamide, 6-hydroxy-2-benzothiazole-sulfonamide 6-pivaloyloxy-2-benzothiazolesulfonamide and the like and mixtures thereof;

(11) anti-fungal agents, such as amphotericin B, nystatin, flucytosine, natamycin, and miconazole and the like and mixtures thereof;

(12) pain-relieving and anesthetic agents, such as etidocaine, cocaine, benoxinate, dibucaine dydrochloride, dyclonine hydrocholoride, naepaine, phenacaine hydrochloride, piperocaine, proparacaine hydrochloride, tetracaine hydrochloide, hexylcaine, bupivacaine, lidocaine, mepivacaine and prilocalne and the like and mixtures thereof;

(13) ophthalmic diagnostic agents, such as

(a) those used to examine the retina, such as choride-sodium fluorescein and the like and mixtures thereof;

(b) those used to examine the conjunctiva, cornea and lacrimal structures, such as fluorescein and rose Bengal and the like and mixtures thereof; and

(c) those used to examine abnormal pupillary responses such as methacholine, cocaine, adrenaline, atropine, hydroxyamphetamine and pilocarpine and the like and mixtures thereof;

(14) ophthalmic agents used as adjuncts in surgery, such as alpha-chymotrypsin, and hyaluronidase and the like; visco-elastic agents, such ass hyaluronates and the like and mixtures thereof;

(15) chelating agents, such as ethylenediamine tetraacetate (EDTA) and deferoxamine and the like; and mixtures thereof;

(16) immunosuppressive agents and anti-metabolites, such as methotrexate, cyclophosphamide, 6-mercaptopurine, cyclosporins such A through I and azathioprine and the like; and mixtures thereof;

(17) angiostatic agents;

(18) muco-secretogogue agents;

(19) proteins and growth factors such as epidermal growth factor;

(20) vitamins and vitamin derivatives such as vitamins A, B12, C, D, E, folic acid and their derivatives;

(21) combinations of the above such as antibiotic/anti-inflammatory as in neomycin sulfate-dexamethasone sodium phosphate, quinolone-NSAID and the like; and concomitant anti-glaucoma therapy, such as timolol maleate-aceclidine and the like.

When a therapeutic component is present in the compositions of the present invention, the amount of such therapeutic component in the composition preferably is effective to provide the desired therapeutic effect to the human or animal to whom the composition is administered.

Typically, when a therapeutic component is present, the compositions comprising oil-in-water emulsions of the present invention contain from or at least about 0.001%, for example, about 0.01%, to about 5% (w/v) of the therapeutic component, e.g., medicament or pharmaceutical, on a weight to weight basis. Thus, for example, from one drop of liquid composition which contains about 25 mg of composition, one would obtain about 0.0025 mg to about 1.25 mg of therapeutic component.

The particular therapeutic component, e.g., drug or medicament, used in the pharmaceutical compositions of this invention is the type which a patent would require or benefit from for the treatment, e.g., pharmacological treatment, of a condition which the patient has or is to be protected from or from which the patient is suffering. For example, if the patient is suffering from glaucoma, the drug of choice may be timolol and/or one or more other anti-glaucoma components.

It is within the knowledge of one skilled in the art to determine the correct amounts of therapeutic component, e.g., drug, to be added to a composition of the invention in order to assure the efficacious delivery of the desired therapeutic component.

Another aspect of this invention is the use of the herein described compositions comprising oil-in-water emulsions for the treatment of dry eye. For this use, one would administer a composition as needed as determined by one skilled in the art. For example, ophthalmic demulcents such as carboxymethylcellulose, other cellulose polymers, dextran 70, gelatin, glycerine, polyethylene glycols (e.g., PEG 300 and PEG 400), polysorbate 80, propylene glycol, polyvinyl alcohol, povidone and the like and mixtures thereof, may be used in the present ophthalmic compositions, for example, compositions useful for treating dry eye.

In another embodiment, the present compositions are useful as multi-purpose care compositions, rigid gas permeable soaking and conditioning solutions, rewetting compositions and cleaning compositions, for example, in-the-eye cleaners, for contact lens care.

All types of contact lenses may be cared for using compositions of the present invention. For example, the contact lenses may be soft, rigid and soft or flexible gas permeable, silicone hydrogel, silicon non-hydrogel and conventional hard contact lenses.

A multi-purpose composition, as used herein, is useful for performing at least two functions, such as cleaning, rinsing, disinfecting, rewetting, lubricating, conditioning, soaking, storing and otherwise treating a contact lens, while the contact lens is out of the eye. Such multi-purpose compositions preferably are also useful for re-wetting and cleaning contact lenses while the lenses are in the eye. Products useful for re-wetting and cleaning contact lenses while the lenses are in the eye are often termed re-wetters or “in-the-eye” cleaners. The term “cleaning” as used herein includes the loosening and/or removal of deposits and other contaminants from a contact lens with or without digital manipulation and with or without an accessory device that agitates the composition. The term “re-wetting” as used herein refers to the addition of water over at least a part, for example, at least a substantial part, of at least the anterior surface of a contact lens.

Although the present compositions are very effective as multi-purpose contact lens care compositions, the present compositions, with suitable chemical make-ups, can be formulated to provide a single contact lens treatment. Such single treatment contact lens care compositions, as well as the multi-purpose contact lens care compositions are included within the scope of the present invention.

Methods for treating a contact lens using the herein described compositions are included within the scope of the invention. In general, such methods comprise contacting a contact lens with such a composition at conditions effective to provide the desired treatment to the contact lens.

The contact lens can be contacted with the composition, often in the form of a liquid aqueous medium, by immersing the lens in the composition. During at least a portion of the contacting, the composition containing the contact lens can be agitated, for example, by shaking the container containing the composition and contact lens, to at least facilitate the contact lens treatment, for example, the removal of deposit material from the lens. Before or after such contacting step, in contact lens cleaning, the contact lens may be manually rubbed to remove further deposit material from the lens. The cleaning method can also include rinsing the lens prior to or after the contacting step and/or rinsing the lens substantially free of the composition prior to returning the lens to the wearer's eye.

In addition, methods of applying or administering artificial tears, washing eyes and irrigating ocular tissue, for example, before, during and/or after surgical procedures, are included within the scope of the present invention. The present compositions, as described elsewhere herein, are useful as artificial tears, eyewash and irrigating compositions which can be used, for example, to replenish/supplement natural tear film, to wash, bathe, flush or rinse the eye following exposure to a foreign entity, such as a chemical material or a foreign body or entity, or to irrigate ocular tissue subject to a surgical procedure. Foreign entities in this context include, without limitation, one or more of pollen, dust, ragweed and other foreign antigens, which cause adverse reactions, such as allergic reactions, redness, itching, burning, irritation, and the like in the eye.

The present compositions, having suitable chemical make-ups, are useful in each of these, and other, in-the-eye applications. These compositions can be used in in-the-eye applications in conventional and well-known manners. In other words, a composition in accordance with the present invention can be used in an in-the-eye application in a substantially similar way as a conventional composition is used in a similar application. One or more of the benefits of the present compositions, as discussed elsewhere herein, are provided as the result of such in-the-eye use.

A cleaning component may be included in the present compositions useful to clean contact lenses. When present, the cleaning component should be present in an amount effective to at least facilitate removing, and preferably effective to remove, debris or deposit material from a contact lens.

In one embodiment, cleaning surfactants are employed. A cleaning component can be provided in an amount effective to at least facilitate removing deposit material from the contact lens. Types of deposit material or debris which may be deposited on the lens include proteins, lipids, and carbohydrate-based or mucin-based debris. One or more types of debris may be present on a given lens.

The cleaning surfactant component employed may be selected from surfactants conventionally employed in the surfactant cleaning of contact lenses. Among the preferred surfactants are non-ionic surfactants such Pluronic and Tetronic series surfactants, both of which are block copolymers of propylene oxide and ethylene oxide, available from BASF Corp. Performance Chemicals, Mount Olive, N.J., and the like, for example, one or more vitamin derivative components, for example, vitamin E TPGS (D-alpha-tocopheryl polyethylene glycol 1000 succinate).

In one embodiment, a composition in accordance with the present invention containing such a cleaning surfactant component has a surfactant concentration of between about 0.01 and 1.00 w/v%. However, higher or lower amounts may be used.

The present compositions may further comprise one or more antimicrobial agents (i.e., preservatives or disinfectants) to preserve the compositions from microbial contamination and/or disinfect contact lenses. The amount of the disinfectant component present in the liquid aqueous medium is effective to disinfect a contact lens placed in contact with the composition.

In one embodiment, for example, when a multi-purpose contact lens composition is desired, the disinfectant component includes, but is not limited to, quaternary ammonium salts used in ophthalmic applications such as poly[dimethylimino-w-butene-1,4-diyl] chloride, alpha-[4-tris(2-hydroxyethyl)ammonium]-dichloride (chemical registry number 75345-27-6, available under the trademark Polyquatemium 1} from Onyx Corporation), poly (oxyethyl (dimethyliminio)ethylene dmethyliminio) ethylene dichloride sold under the trademark WSCP by Buckman laboratories, Inc. in Memphis, Tenn., benzalkonium halides, salts of alexidine, alexidine-free base, salts of chlorhexidine, hexetidine, alkylamines, alkyl di- and tri-amine, tromethamine (2-amino-2-hydroxymethyl-1,3 propanediol), hexamethylene biguanides and their polymers, antimicrobial polypeptides, and the like and mixtures thereof. A particularly useful disinfectant component is selected from one or more (mixtures) of polyhexamethylene biguanide (PHMB), Polyquatemium-1, ophthalmically acceptable salts thereof, and the like and mixtures thereof.

The salts of alexidine and chlorhexidine can be either organic or inorganic and are typically disinfecting gluconates, nitrates, acetates, phosphates, sulphates, halides and the like. Generally, the hexamethylene biguanide polymers, also referred to as polyaminopropyl biguanide (PAPB), have molecular weights of up to about 100,000. Such compounds are known and are disclosed in U.S. Pat. No. 4,758,595 which is incorporated in its entirety by reference herein.

The disinfectant components useful in the present invention are preferably present in the present compositions in concentrations in the range of about 0.00001% to about 2% (w/v).

More preferably, the disinfectant component is present in the present compositions at an ophthalmically acceptable or safe concentration such that the user can remove the disinfected lens from the composition and thereafter directly place the lens in the eye for safe and comfortable wear.

When a contact lens is desired to be disinfected by a disinfectant component, an amount of disinfectant effective to disinfect the lens is used. Preferably, such an effective amount of the disinfectant reduces the microbial burden on the contact lens by one log order, in three hours. More preferably, an effective amount of the disinfectant reduces the microbial load by one log order in one hour.

The disinfectant component is preferably provided in the present composition, and is more preferably soluble in the aqueous component of the present composition.

The present compositions may include an effective amount of a preservative component. Any suitable preservative or combination of preservatives may be employed. Examples of suitable preservatives include, without limitation, Purogene®, polyhexamethylene biguanide (PHMB), Polyquatemium-1, ophthalmically acceptable salts thereof, and the like and mixtures thereof, benzalkonium chloride, methyl and ethyl parabens, hexetidine and the like and mixtures thereof. The amount of preservative components included in the present compositions are such to be effective in preserving the compositions and can vary based on the specific preservative component employed, the specific composition involved, the specific application involved, and the like factors. Preservative concentrations often are in the range of about 0.00001% to about 0.05% or about 0.1% (w/v) of the composition, although other concentrations of certain preservatives may be employed.

Very useful examples of preservative components in the present invention include, but are not limited to, chlorite components. Specific examples of chlorite components useful as preservatives in accordance with the present invention include stabilized chlorine dioxide (SCD), metal chlorites, and the like and mixtures thereof. Technical grade (or USP grade) sodium chlorite is a very useful preservative component. The exact chemical composition of many chlorite components, for example, SCD, is not completely understood. The manufacture or production of certain chlorite components is described in McNicholas U.S. Pat. No. 3,278,447, which is incorporated in its entirety by reference herein. Specific examples of useful SCD products include that sold under the trademark Dura Klor by Rio Linda Chemical Company, Inc., that sold under the trademark Anthium Dioxide® by International Dioxide, Inc. North Kingstown, R.I., that sold under the trademark Camebon 200® by International Dioxide, Inc and Purogene® by BioCide International, Norman, Okla. (also known as Purite®, available from Allergan, Inc.).

Other useful preservatives include antimicrobial peptides. Among the antimicrobial peptides which may be employed include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

The compositions of the present invention may include viscosity modifying agents or components, such as cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose; carbomers (e.g. carbopol. RTM); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

It is desirable in some instances to include sequestering agents or components in the present compositions in order to, and in an amount effective to, bind metal ions, which, for example, might otherwise stabilize cell membrances of microorganisms and thus interfere with optimal disinfection activity. Alternatively, it is desirable in some instances to bind metal ions to prevent their interaction with other species in the compositions. Sequestering agents are included, if at all, in amounts effective to bind at least a portion, for example, at least a major portion of the metal ions present. Such sequestering components usually are present in amounts ranging from about 0.01 to about 0.2 w/v%. Examples of useful sequestering components include, without limitation ethylene-diaminetetraacetic acid (EDTA) and its potassium or sodium salts and low molecular weight organic acids such as citric and tartaric acids and their salts, e.g., sodium salts.

The present compositions may comprise effective amounts of one or more additional components. For example, one or more conditioning components or one or more contact lens wetting agents and the like and mixtures thereof may be included. Acceptable or effective concentrations for these and other additional components in the compositions of the invention are readily apparent to the skilled practitioner.

Each of the components may be present in either a solid or liquid form of the present compositions. When the additional component or components are present as a solid, they can either be intimately admixed such as in a powder or compressed tablet or they can be substantially separated, although in the same particles, as in an encapsulated pellet or tablet. The additional component or components can be in solid form until desired to be used, whereupon they can be dissolved or dispersed in the aqueous component of the present composition in order to, for example, effectively contact the surface of a contact lens.

When any component is included, it is preferably compatible under typical use and storage conditions with the other components of the composition.

It will be understood by those of skill in the art that numerous and various modifications can be made without departing from the spirit of the present invention. Therefore, it should be clearly understood that the following examples are illustrative only and are not intended to limit the scope of the present invention.

### EXAMPLES

### Example 1

Method of Preparing Ophthalmic Solution

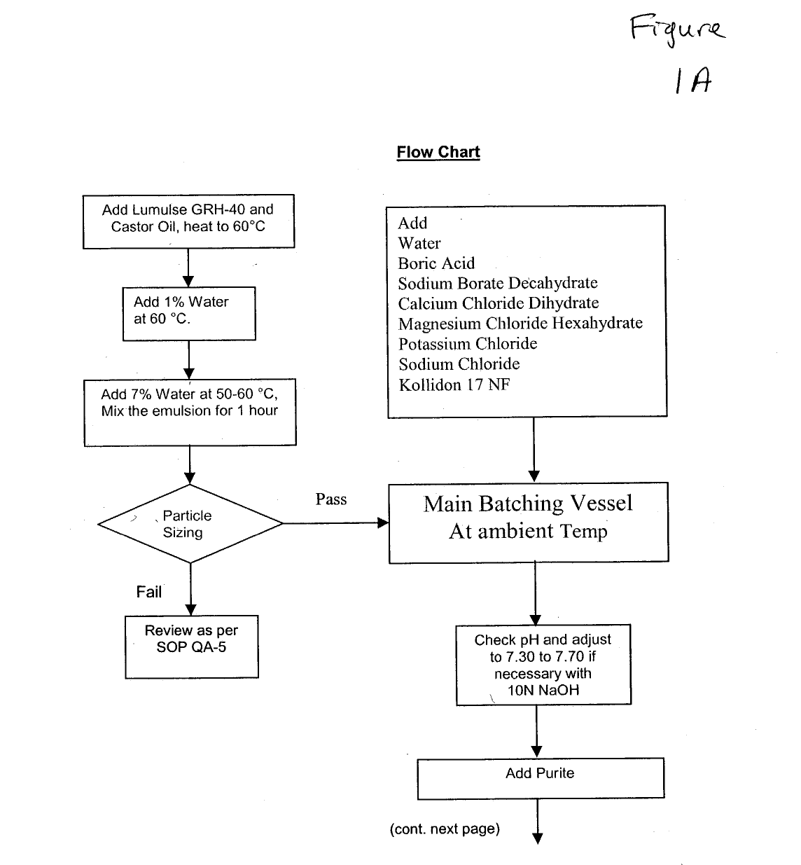

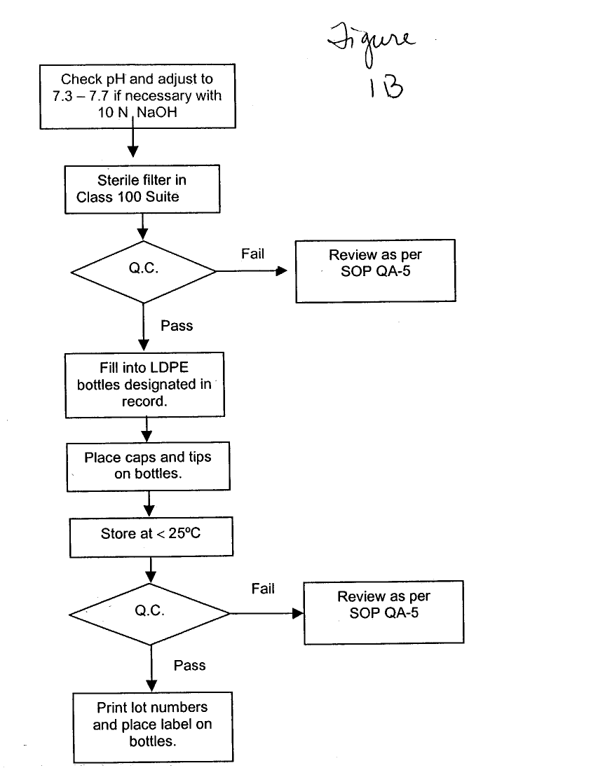

The following example will be described with respect to a one-component surfactant system. In this example, PEG-40 hydrogenated castor oil, a 40 mole ethoxylated derivative of hydrogenated castor oil, is exemplified. Reference is made to FIG. 1 and Table 1. FIG. 1 shows a flow chart for the method. Table 1 shows amounts of the various components for this example.

PEG-40 hydrogenated castor oil (Lumulse GRH-40, Lambent Technologies Corp., Skokie, Ill.) and castor oil were heated. The temperature must be high enough that all components are in the liquid state but not so high as to jeopardize the stability of the components. In the present example, a temperature of 60+/−2° C. was used.

A small amount of the total water (1%) was added at 60+/−2° C., to form a transparent white paste. The paste was mixed for until the mixture was homogenous. After the paste was formed, more water was added to the paste between 50-62° C. In this example, 7% of the total water was added and mixing was carried out for 1 hour at 200-1000 rpm until the mixture was homogeneous. At this stage, an emulsion concentrate had formed.

The particles (droplets) were then sized using a Horiba LA-920 particle size analyzer according to the manufacturer's instructions. Preferably, the particles are between 0.08 and 0.18 microns in size before passing to the next step.

The emulsion concentrate was mixed with a separately prepared solution of the remaining water, buffer, electrolytes (calcium chloride dihydrate, magnesium chloride hexahydrate, potassium chloride and sodium chloride) and Kollidon 17 NF (see Table 1) for about 30 minutes. While the electrolytes are not necessary to form the emulsions, they are very helpful to preserve ocular tissue integrity by maintaining the electrolyte balance in the eye. Likewise, the buffer is not critical to form the emulsion, but is necessary to properly maintain a compatible ocular pH. A boric acid/sodium borate buffer system is preferred because a phosphate-based buffer system will precipitate with the electrolytes.

ThepHwas adjusted to 7.35 to 7.55 with 10N NaOH, if necessary. Note that this pH range is optimal for tissue maintenance and to avoid ocular irritation. This is also the optimal pH range for stability of Purogene® which was added as a preservative. Purogeneg was then added according to the calculation shown in Table 1. Thereafter, pH was checked and adjusted to pH 7.5+/−0.2 if necessary with 10N NaOH. Note that the pH may only be adjusted with a base such as 10 N NaOH after the addition of Purogene®, as high local solution concentrations of acid formed during acid pH adjustment will cause destruction of the Purogene®.

In the next step, the emulsion was stored covered in the dark at less than 25° C. until sterile filtered. Maximum storage time is 72 hours.

The composition is then filter sterilized using a 0.22 micron filter. Preferably, 98-99% of the emulsion should pass through the 0.22 micron filter. Note that particles larger than 0.22 micron may pass through by altering their shape temporarily. The material was then tested to verify the effectiveness of the sterilization step. The material was hten bottled and stored. Pre-fill release specifications for this example were pH 7.3-7.7, mean particle size of 0.09-0.17 microns and physical appearance of a milky white solution. Post-fill release specifications were pH 7.3-7.7, potential chlorine dioxide of 60-70 ppm, castor oil 1.1-1.4% (w/w), Kollidon 17 NF 0.2-0.4% (w/w), osmolality 250-280 mOsm/kg, and sterility USP.

TABLE 1
EMULSION FORMULATION FOR EXAMPLE 1

| Ingredient/Component | Amount/1000 g |
|---|---|
| Lumulse GRH-40 | 10 |
| Castor oil | 12.5 |
| Boric Acid | 6.0 |
| Sodium Borate | 0.35 |
| Calcium Chloride dihydrate | 0.06 |
| Magnesium Chloride hexahydrate | 0.06 |
| Potassium Chloride | 1.4 |
| Sodium Chloride | 3.5 |
| Kollidon 17 PF | 3.0 |
| 10 N Sodium Hydroxide | pH adjust |
| Purogene ® | see below¹ |
| Purified Water, USP | see below² |
| Sterile filter, 0.22 micron | |

¹Purogene ® calculation: the amount of raw material to be added must be calculated on the basis of the assay of the raw material lot.
0.0065% (w/w) × 1000 g = grams of Purogene ® raw material Purogene ® raw material assay value % (w/w) required per 1000 g
Purogene ® (g) required per 1000 g/1000 g × Batch size (g) = Purogene ® (g) required/batch size
²Water amount calculation per 1000 g
The amount of water to be added must be calculated on the basis of the amount of Purogene ® raw material to be added.
Water (g) per 1000 g = 963.13 − Purogene ® (g) required per 1000 g

### EXAMPLE 2

Neutral Red Retention Assay for Evaluation of Cytotoxicity of Ophthalmic Emulsions.

Cytotoxicity of solutions was evaluated with a standard Neutral Red Retention Assay. The relevance of the neutral red retention cytotoxicity assay is based upon established observations that certain materials that are irritating or damaging to tissues such as ocular tissues in vivo are cytotoxic to certain cell types in vitro, and the degree of irritation or damage correlates with the level of cytotoxicity. In healthy and viable cells, neutral red dye is incorporated and stored in the lysosomes of the cell. Upon damage to the cellular membrane, the neutral red dye is released from the lysosomes. The level of membrane damage inversely correlates to the amount of neutral red still retained by the cell. Extraction of the dye from the cells after exposure to the test agents evaluates the integrity of the cellular membrane and degree of cytotoxicity induced.

Madin-Darby Canine Kidney (MDCK) cells were used in the assay. Cells were added to each well of 96 well flat bottom tissue culture plates at 1×10⁴ cells/well in 200 microliters complete medium. Complete medium is Dulbecco's Modified Eagle's Medium (DMEM) complete growth medium with 10% fetal bovine serum. Cells were incubated to confluence in 3-4 days at 37° C./5% CO₂. Media was decanted and blotted from the plate wells. Neutral red (200 microliters) at a final concentration of 50 micrograms per ml in complete medium was added to each well and incubated for 3 hours at 37° C./5% CO₂. The neutral red solution was decanted and blotted from the plate wells The wells were washed 1× with 100 microliters/well with Dulbecco's Phosphate Buffered Saline with Ca⁺⁺ and Mg⁺⁺ (DPBS). The DPBS was decanted and blotted and 100 microliters of test or control solution was added to the wells. Each solution was added to at least 6 wells in a single column, with the outer wells on each plate receiving only DPBS as a control. Separate plates were designated for each contact time point. Plates were incubated at 37° C./5% CO₂ for the designated contact time. The time points generally tested are 15, 30, 60, 90, 120 and 180 minutes. Plates were removed from the incubator at each time point, decanted and blotted, and then washed 1× with 100 microliters/well of DPBS, decanted and blotted. Next, 100 microliters/well of the neutral red “wash/fix” solution was added and allowed to stand at room temperature at ambient conditions for 5 minutes. The neutral red “wash/fix” solution was 1% formalin, 1% CaCl₂ (w/v) and 98% distilled water. The fixative was decanted and blotted and 100 microliters/well of solvent solution was added. Solvent solution was 1% acetic acid, 50% ethanol and 49% distilled water. The plates were allowed to extract with the solvent solution at room temperature at ambient conditions on a plate shaker (low speed) for 10 minutes. Thereafter, the plates were read at 540 nm on a microliter plate spectrophotometer. Absorbance readings for all wells for each sample or control were averaged and the results as percent neutral red retained compared to the DPBS control were calculated ((Ave O.D. of test sample/Ave O.D. of control)×100=% of control). Test results were plotted graphically as neutral red retention (% of control), Y, vs time of exposure (minutes), X.

### EXAMPLE 3

Formulations for Cytotoxicity Studies

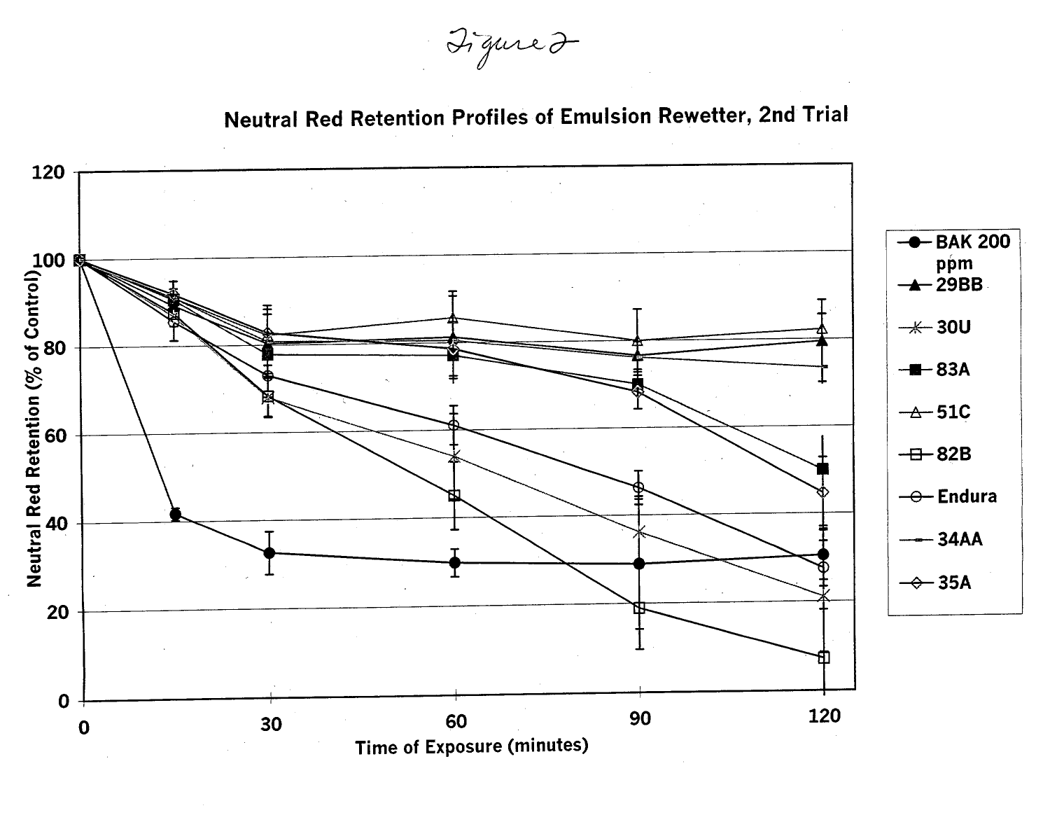

The Formulations shown in Table 2 were prepared essentially as described in Example 1. Formulations 29BB, 51C, 82B, 34AA and 35A with the detergent Tween 80 were compared to formulations 30U and 83U which were prepared without Tween 80. As can be seen by the data of FIG. 2, the presence or absence of the Tween 80 detergent did not materially effect the cytotoxicity. Formulations 29BB and 34AA were prepared without Purogene® and amounts of polyethoxylated hydrogenated castor oil, GRH-40, were also varied slightly without major effects. Formulas 29BB and 51C differ only in Purogene® concentration, and yet have essentially identical neutral red retention at 120 min, 79 and 82%, respectively.

Formulations 30U and 82B differ from the other formulations in Table 2 in that they contained glycerin and not NaCl. The Enduram formulation also contains glycerin. As can be seen in FIG. 2, the glycerin-containing formulations were the most cytotoxic.

The pH was varied from 7.19 to 7.75. Formulas 51C and 35A differ only in pH, with 51C having a pH of 7.39 and 35A a pH of 7.75. Their respective neutral red retention values at 120 minutes were 82 and 45%, indicating a substantial cytotoxic effect due to pH 7.75. Osmolarity ranged from 230-286. Particle size was fairly constant. All formulations were compared to Endura™.

TABLE 2
FOR EXAMPLE 3

| | Formulation | | | | | | | |
|---|---|---|---|---|---|---|---|---|
| | | | | | | ENDURA | | |
| | 29BB | 30U | 83A | 51C | 82B | (TM)* | 34AA | 35A |
| Tween-80 | 0.25 | | | 0.25 | 0.25 | 1 | 0.25 | 0.25 |
| GRH-40 | 0.75 | 1 | 1 | 0.75 | 0.75 | 0 | 0.75 | 0.75 |
| Castor Oil | 1.25 | 1.25 | 1.25 | 1.25 | 1.25 | 1.25 | 1.25 | 1.25 |
| Boric Acid | 0.6 | 0.6 | 0.6 | 0.6 | 0.6 | 0 | 0.6 | 0.6 |
| Sodium Borate | 0.035 | 0.035 | 0.035 | 0.035 | 0.035 | 0 | 0.035 | 0.035 |
| CaCl₂ 2H₂0 | 0.006 | 0.006 | 0.006 | 0.006 | 0.006 | 0 | 0.006 | 0.006 |
| MgCl₂ 6H₂0 | 0.006 | 0.006 | 0.006 | 0.006 | 0.006 | 0 | 0.006 | 0.006 |
| KCl | 0.14 | 0.14 | 0.14 | 0.14 | 0.14 | 0 | 0.14 | 0.14 |
| NaCl | 0.25 | | 0.25 | 0.25 | | 0 | 0.25 | 0.25 |
| Glycerin | | 1 | | | 1 | 1 | | |
| Purogene ®, ppm | 0 | 79.8 | 79.7 | 79.9 | 79.9 | 0 | 0 | 79.9 |
| pH | 7.39 | 7.19 | 7.68 | 7.39 | 7.68 | 7.33 | 7.39 | 7.75 |
| Osmo | 286 | 281 | 230 | 286 | 263 | 240 | 286 | 286 |
| Particle size (μm) | 0.14 | 0.13 | 0.14 | 0.14 | 0.15 | | 0.14 | 0.14 |
| 99% Cumulative (μm) | 0.25 | 0.25 | 0.28 | 0.25 | 0.29 | | 0.25 | 0.25 |

| All components in w/w % except Purogene ® | | same as | 51C w/ |
|---|---|---|---|
| Notes | 51C w/out | 29BB | pH 7.75 |
| | Purogene ® | | |

*Endura (TM) additionally contains another non-ionic osmolyte and contains a polymer to adjust viscosity. The particle size of Endura ™ is substantially larger than 0.15 microns and less than 1.0 micron.

### EXAMPLE 4

Formulations for Cytotoxicity Studies

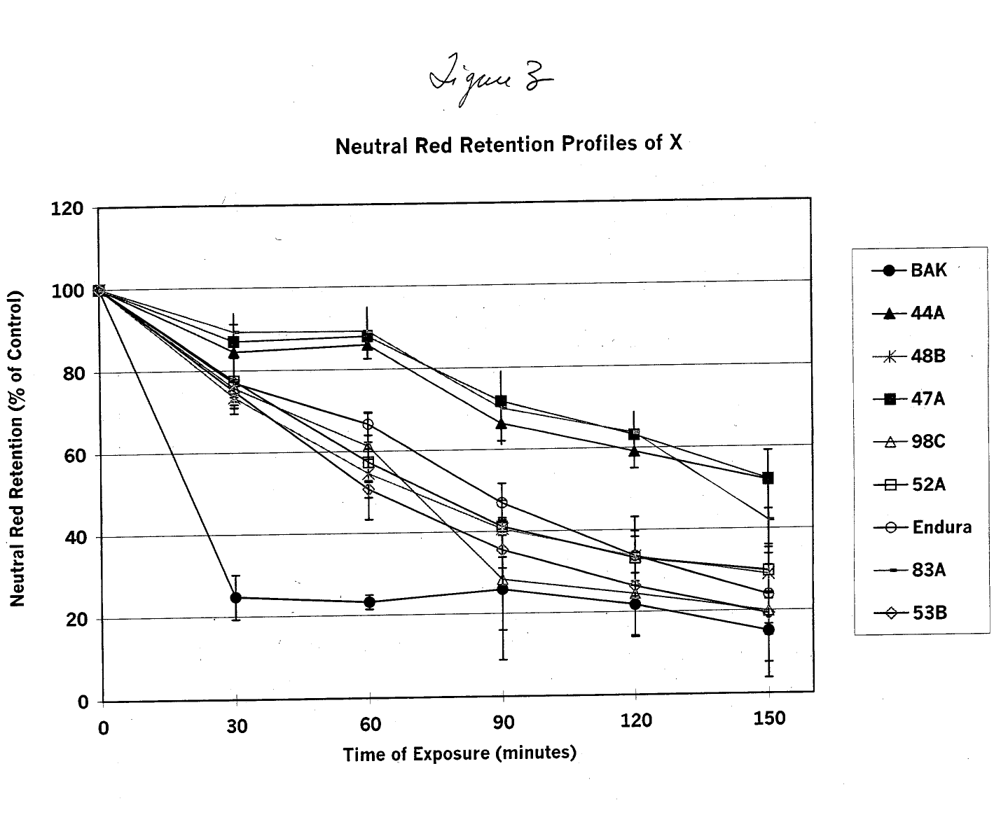

The Formulations shown in Table 3 were prepared essentially as described in Example 1. FIG. 3 shows the cytotoxicity data for the the Formulations of Table 3. Specifically, the effects of osmolality, Tween 80 and pH were tested. Solutions 48B, 52A and 53B, containing Povidone, PEG300 and CMC, respectively, each had osmolalities of 163-167 mOsm/kg, which evidently was responsible for producing the observed cytotoxicity, since these polymers are all considered to be non-cytotoxic. Solutions 44A and 47A differ only in Tween 80. Their respective neutral red retention values at 120 minutes were 59 and 63%. Solutions 44A and 83A differ only in pH, 7.35 vs 7.68, respectively. Their respective neutral red retention values at 120 minutes were 59 and 64%, indicating no effect of pH 7.68 in this experiment.

TABLE 3
FOR EXAMPLE 4
FORMULATIONS RUN IN CYTOTOXICOLOGY EXPERIMENT

| | Formulation | | | | | | | |
|---|---|---|---|---|---|---|---|---|
| | 44A | 48B | 47A | 98C | 52A | Endura | 83A | 53B |
| | Description | | | | | | | |
| | 83A with | | 51C with | 30U no | | | | |
| | pH 7.35 | Povidone | pH 7.37 | Purogene ® | PEG300 | As Before | As Before | with CMC |
| Tween-80 | | | 0.25 | | | | | |
| GRH-40 | 1 | 1 | 0.75 | 1 | 1 | | 1 | 1 |
| Castor Oil | 1.25 | 1.25 | 1.25 | 1.25 | 1.25 | | 1.25 | 1.25 |
| Boric Acid | 0.6 | 0.6 | 0.6 | 0.6 | 0.6 | | 0.6 | 0.6 |
| Sodium Borate | 0.035 | 0.035 | 0.035 | 0.035 | 0.035 | | 0.035 | 0.035 |
| CaCl₂ 2H₂0 | 0.006 | 0.006 | 0.006 | 0.006 | 0.006 | | 0.006 | 0.006 |
| MgCl₂ 6H₂0 | 0.006 | 0.006 | 0.006 | 0.006 | 0.006 | | 0.006 | 0.006 |
| KCl | 0.14 | 0.14 | 0.14 | 0.14 | 0.14 | | 0.14 | 0.14 |
| NaCl | 0.25 | 0.040 | 0.25 | glycerin1.00 | 0.020 | | 0.25 | 0.01 |
| CMC | | | | | | | | Low Visc 0.5 |
| Povidone | | 0.15 | | | | | | |
| Peg 300 | | | | | 0.3 | | | |
| Purogene ®, ppm | 70.26 | 70.11 | 70.00 | 0.00 | 69.96 | | 79.72 | 69.79 |
| pH | 7.35 | 7.36 | 7.37 | 7.74 | 7.38 | | 7.68 | 7.393 |
| Osmolality | 233 | 163 | | 293 | 167 | | 230 | 163 |
| Particle size ave | 0.120 | 0.120 | 0.162 | 0.149 | 0.120 | | 0.145 | 0.120 |
| 99% Cumulat | 0.264 | 0.264 | 0.299 | 0.28 | 0.264 | | 0.284 | 0.264 |

Amounts are given in w/w % unless otherwise noted.

### EXAMPLE 5

Formulations for Cytotoxicity Studies

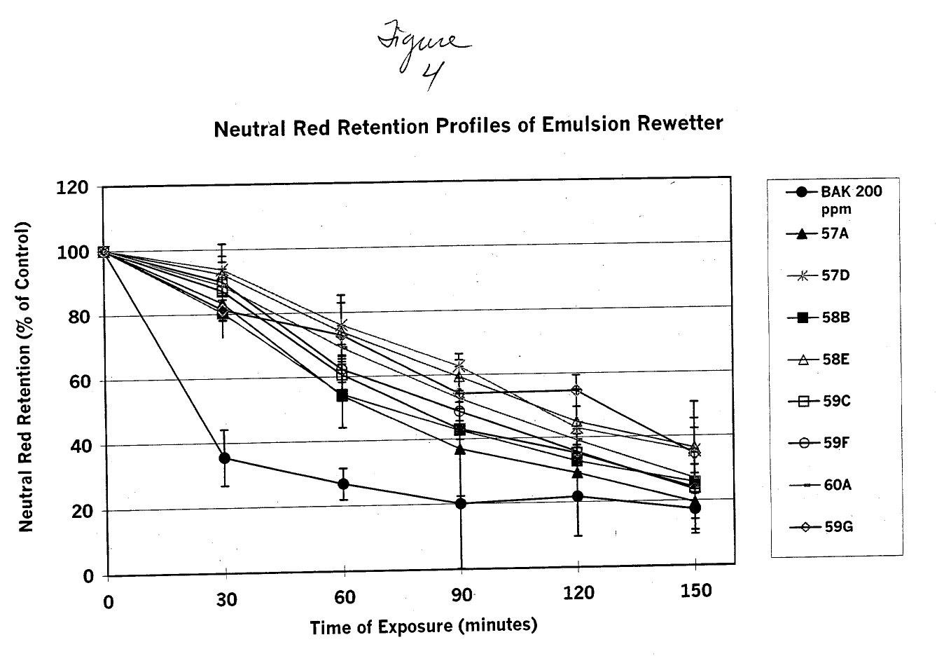

The Formulations shown in Table 4 were prepared essentially as described in Example 1. FIG. 4 shows the cytotoxicity data for the the Formulations of Table 4. For Table 4, amounts are given in grams per 1000 g unless otherwise noted. Cytotoxicities were similar for all solutions. Osmolality differences are believed to account for observed differences, with greater cytotoxicity associated with lower osmolality.

TABLE 4
FORMULATIONS FOR EXAMPLE 5

| INGREDIENT/ | | | | | | | | |
|---|---|---|---|---|---|---|---|---|
| COMPONENT | 57A | 57D | 58B | 58E | 59C | 59F | 60A | 59G |
| GRH-40 | 10 | 10 | 10 | 10 | 10 | 10 | 10 | 10 |
| Castor Oil | 12.5 | 12.5 | 12.5 | 12.5 | 12.5 | 12.5 | 12.5 | 12.5 |
| Boric Acid | 6.0 | 6.0 | 6.0 | 6.0 | 6.0 | 6.0 | 6.0 | 6.0 |
| Sodium Borate | 0.35 | 0.35 | 0.35 | 0.35 | 0.35 | 0.35 | 0.35 | 0.35 |
| CaCl2*2H2O | 0.06 | 0.06 | 0.06 | 0.06 | 0.06 | 0.06 | 0.06 | 0.06 |
| MgCl2*6H2O | 0.06 | 0.06 | 0.06 | 0.06 | 0.06 | 0.06 | 0.06 | 0.06 |
| KCl | 1.4 | 1.4 | 1.4 | 1.4 | 1.4 | 1.4 | 1.4 | 1.4 |
| NaCl | 1.10 | 1.62 | 0.88 | 1.12 | 0.60 | 0.91 | 1.27 | 2.06 |
| CMC (low viscosity) | | | | | 5.0 | 5.0 | 5.0 | 5.0 |
| Povidone | 1.5 | 1.5 | | | | | | |
| PEG 300 | | | 3.0 | 3.0 | | | | |
| Purogene ®, ppm | 70.0 | 70.0 | 70.1 | 70.1 | 69.7 | 69.7 | 69.7 | 69.7 |
| pH | 7.40 | 7.40 | 7.37 | 7.37 | 7.39 | 7.39 | 7.39 | 7.39 |
| Osmo | 188 | 231 | 191 | 231 | 183 | 195 | 205 | 232 |

Ingredients are grams/1000 g unless otherwise noted. Mean particle size for all fomulations was 0.120 microns. Cumulative (99%) particle size was 0.264 microns.

### EXAMPLE 6

Formulations for Cytotoxicity Studies: Effects of CMC, Povidone, and PEG-300

The Formulations shown in Table 5 were prepared essentially as described in Example 1. For Table 5, amounts are given in grams per 1000 g unless otherwise noted.

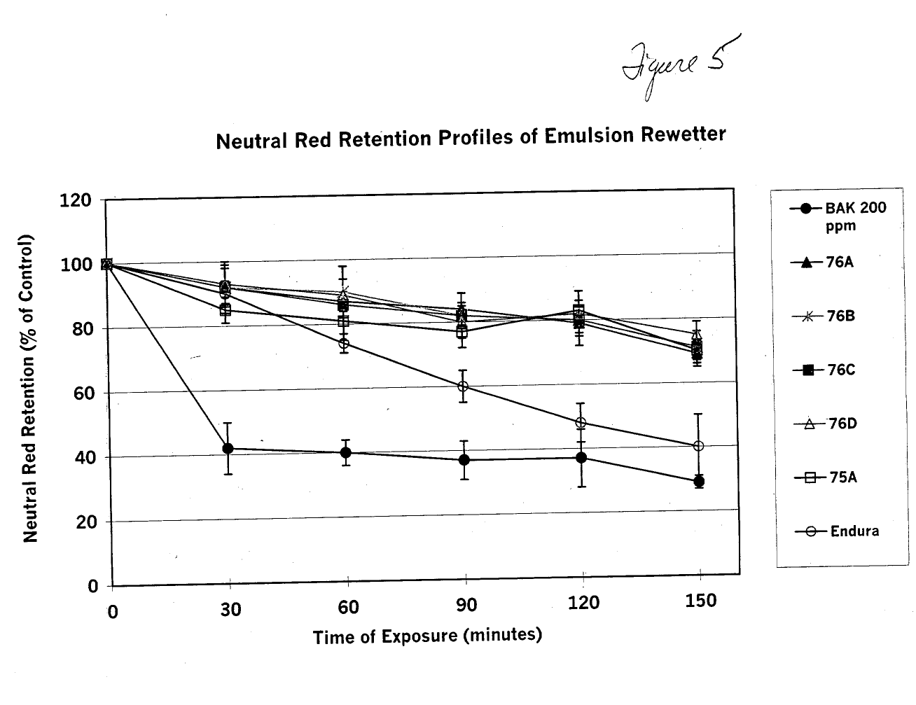

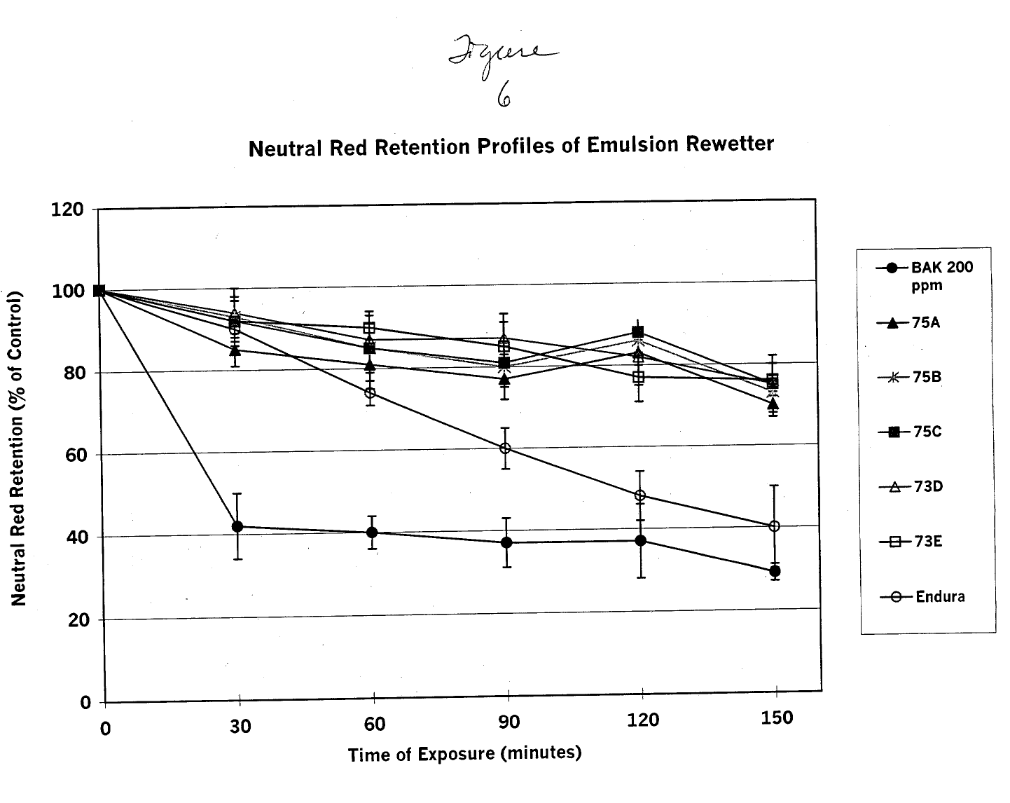

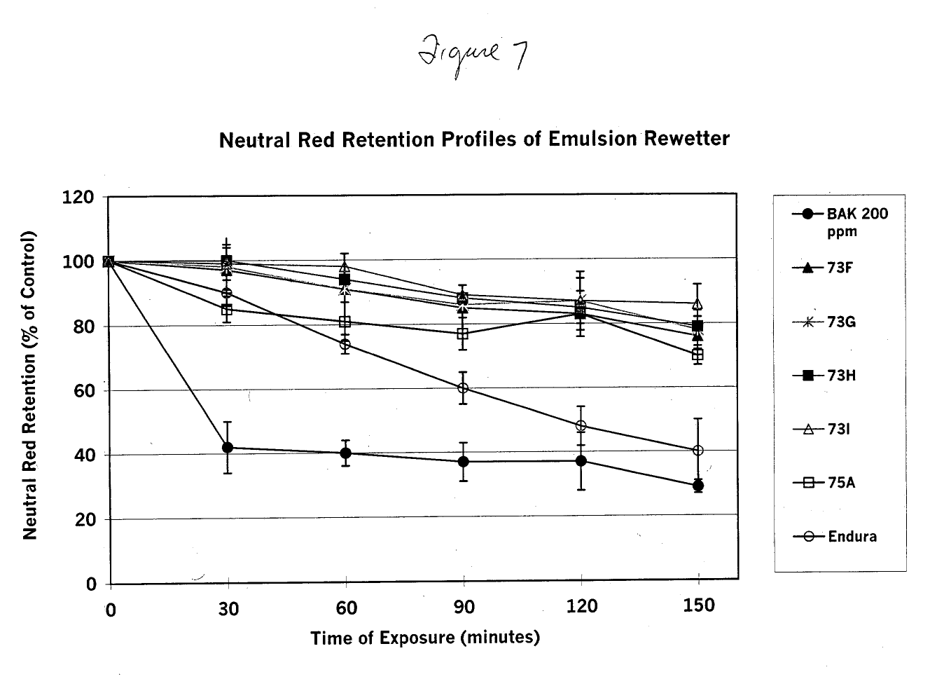

FIGS. 5-7 show the cytotoxicity data for the the Formulations of Table 5. Formulations 76A-D were prepared with CMC (FIG. 5). Formulations 75B & C and 73D & E were prepared with Povidone (FIG. 6). Formulations 73F, G, H, and I were prepared with PEG-300 (FIG. 7). All formulas except 75A contained the additional preservative, WSCP. None of these changes materially affected cytotoxicity.

TABLE 5
FORMULATIONS

| INGRE- | | | | | | | | | | | | | |
|---|---|---|---|---|---|---|---|---|---|---|---|---|---|
| DIENT/ | | | | | | | | | | | | | |
| COM- | | | | | | | | | | | | | |
| PONENT | 76A | 76B | 76C | 76D | 75A | 75B | 75C | 73D | 73E | 73F | 73G | 73H | 73I |
| GRH-40 | 10 | 10 | 10 | 10 | 10 | 10 | 10 | 10 | 10 | 10 | 10 | 10 | 10 |
| Castor Oil | 12.5 | 12.5 | 12.5 | 12.5 | 12.5 | 12.5 | 12.5 | 12.5 | 12.5 | 12.5 | 12.5 | 12.5 | 12.5 |
| Boric Acid | 6.0 | 6.0 | 6.0 | 6.0 | 6.0 | 6.0 | 6.0 | 6.0 | 6.0 | 6.0 | 6.0 | 6.0 | 6.0 |
| Sodium | 0.35 | 0.35 | 0.35 | 0.35 | 0.35 | 0.35 | 0.35 | 0.35 | 0.35 | 0.35 | 0.35 | 0.35 | 0.35 |
| Borate | | | | | | | | | | | | | |
| CaCl2* | 0.06 | 0.06 | 0.06 | 0.06 | 0.06 | 0.06 | 0.06 | 0.06 | 0.06 | 0.06 | 0.06 | 0.06 | 0.06 |
| 2H2O | | | | | | | | | | | | | |
| MgCl2* | 0.06 | 0.06 | 0.06 | 0.06 | 0.06 | 0.06 | 0.06 | 0.06 | 0.06 | 0.06 | 0.06 | 0.06 | 0.06 |
| 6H2O | | | | | | | | | | | | | |
| KCl | 1.4 | 1.4 | 1.4 | 1.4 | 1.4 | 1.4 | 1.4 | 1.4 | 1.4 | 1.4 | 1.4 | 1.4 | 1.4 |
| NaCl | 2.5 | 2.5 | 2.5 | 2.5 | 2.5 | 2.5 | 2.5 | 2.5 | 2.5 | 2.5 | 2.5 | 2.5 | 2.5 |
| WSCP, ppm | 1 | 2 | 3 | 3 | | 1 | 2 | 3 | 3 | 1 | 2 | 3 | 3 |
| CMC | 5.0 | 5.0 | 5.0 | 5.0 | | | | | | | | | |
| Povidone | | | | | | 1.5 | 1.5 | 1.5 | 1.5 | | | | |
| PEG | | | | | | | | | | 3.0 | 3.0 | 3.0 | 3.0 |
| Purogene ®, | 65 | 65 | 65 | 50 | 65 | 65 | 65 | 65 | 50 | 65 | 65 | 65 | 50 |
| ppm | | | | | | | | | | | | | |
| pH | 7.58 | 7.55 | 7.55 | 7.54 | 7.56 | 7.56 | 7.58 | 7.50 | 7.51 | 7.51 | 7.53 | 7.54 | 7.52 |
| Osmo | 282 | 278 | 277 | 275 | 265 | 268 | 268 | 265 | 261 | 276 | 277 | 275 | 274 |

CMC = Carboxymethyl cellulose;
WSCP = water-soluble cationic polymer
For Table 5, mean particle size was 0.111 microns for all formulations and 99% cumulative was 0.213 microns for all formulations.

### EXAMPLE 7

Summary of Results of Cytotoxicity Studies

The results in FIGS. 2-7 show that the glycerin-containing formulas 30U (pH 7.19), 82B (pH 7.68) and Endura™ (pH 7.33) are all significantly cytotoxic. The cytotoxicity in these formulas is also due to the low and high pH values. The results more clearly show that a shift in pH from 7.39 (51C) to 7.75 (35A) makes the solution more cytotoxic, as expected. However, small pH shifts are well tolerated. The presence of Purogene® in 51C does not enhance cytotoxicity of the Purogene®-free equivalent formulas 29BB and 34AA. Formulas with low osmolality (163-167 mOsm/kg) were cytotoxic. However, smaller changes in solution osmolality did not produce large changes in cytotoxicity. Likewise, GRH-40 alone or in the presence of polysorbate 80 does not effect cytotoxicity significantly. The presence of ophthalmic demulcent polymers such as CMC, Povidone and PEG did not contribute to cytotoxicity. Overall, the results confirm that self-emulsifying oil-in-water emulsions can be constructed from 1 or 2 surfactants such that the solutions are less cytotoxic than a currently marketed oil-in-water ophthalmic emulsion, Endura™, which is manufactured via conventional emulsification methods using a prior art surfactant and viscosity-based emulsion stabilization.

### EXAMPLES 8-21

Additional Formulation Examples.

Examples 8-21 (Tables 6-11) show additional formulations prepared in accordance with the invention. Example 8 particularly exemplifies Cremophor RH-60 as the surfactant/emulsifier produced from ethoxylation of hydrogenated castor oil. Example 9 exemplifies Cremophor RH-40 as the surfactant/emulsifier produced from ethoxylation of hydrogenated castor oil. Example 21 exemplifies TPGS.

TABLE 6

| | Example 8 | Example 9 |
|---|---|---|
| Ingredient | % w/w | % w/w |
| Cremophor RH-60 | 1.75 | |
| Cremophor RH-40 | | 1.5 |
| Castor Oil | 1.25 | 1.25 |
| Balanced Electrolytes | 0.397 | |
| Glycerin | | 1.00 |
| Pemulen TR-2 | | 0.10 |
| Boric Acid | 0.60 | 0.60 |
| Purogene ® (2.15 w/v %) | 0.37 | 0.37 |
| Sodium Hydroxide To adjust | | |
| pH to about 7.4 | | |
| Purified Water | q.s. 100 | q.s. 100 |

TABLE 7

| | Example | Example | Example | Example |
|---|---|---|---|---|
| | 10 | 11 | 12 | 13 |
| Ingredient | % w/w | % w/w | % w/w | % w/w |
| Lumulse GRH-40 | 1 | 1.2 | 1 | 1 |
| Castor Oil | 1.35 | 1.5 | 1.25 | 1.25 |
| Boric Acid | 0.6 | 0.6 | 0.6 | 0.6 |
| Sodium Borate 10H₂0 | 0.035 | 0.035 | | |
| CaCl₂.2H₂O | 0.006 | 0.016 | | |
| MgCl₂.6H₂O | 0.006 | 0.006 | | |
| KCl | 0.14 | 0.14 | | |
| NaCl | 0.25 | 0.25 | | |
| Glycerin | | | 1 | 1 |
| HPMC | | 0.1 | 0.1 | |
| Pemulen TR-2 | | | | 0.10 |
| Purogene ® | 0.37 | 0.37 | 0.37 | 0.37 |
| (2.15 w/v %) | | | | |
| pH | 7.621 | 7.321 | 7.3 | 7.3 |
| Viscosity (cps) | | 40.9 | 41.3 | |
| Osmolality (mOsm) | 230 | 247 | 230 | |
| Particle Mean Size (μm) | 0.14 | 0.14 | 0.14 | |
| 99% Cumulative Size | 0.263 | 0.19 | 0.27 | |
| (μm) | | | | |

TABLE 8

| | Example 14 | Example 15 |
|---|---|---|
| Ingredient | % w/w | % w/w |
| Lumulse GRH-40 | 1.5 | 1.5 |
| Castor Oil | 1.25 | 1.25 |
| Boric Acid | 0.6 | 0.6 |
| Sodium Borate 10H₂O | 0.035 | 0.035 |
| CaCl₂.2H₂0 | 0.006 | 0.006 |
| MgCl₂.6H₂O | 0.006 | 0.006 |
| KCl | 0.14 | 0.14 |
| Glycerin | 1 | 1 |
| HPMC (F4M) | 0.7 | |
| Purogene ® (2.15 w/v %) | 0.37 | 0.37 |
| pH | 7.5 | 7.3 |
| Viscocity (cps) | 64.8 | |
| Osmolality (mOsm) | 271 | |
| Particle Mean Size (um) | 0.33 | |
| 99% Cumulative Size (um) | 0.66 | |

TABLE 9

| | Example | Example | Example | Example |
|---|---|---|---|---|
| | 16 | 17 | 18 | 19 |
| Ingredient | % w/w | % w/w | % w/w | % w/w |
| GRH-40 | 1 | 3.2 | 0.4 | 0.75 |
| Castor Oil | 1.25 | 4 | 1 | 1.25 |
| Tween-80 | | | 0.4 | 0.25 |
| Boric Acid | 0.6 | 0.6 | 0.6 | 0.6 |
| Sodium Borate 10H₂0 | 0.035 | 0.035 | 0.035 | 0.035 |
| CaCl₂.2H₂O | 0.006 | 0.016 | 0.006 | 0.006 |
| MgCl₂.6H₂O | 0.006 | 0.006 | 0.006 | 0.006 |
| KCl | 0.14 | 0.14 | 0.14 | 0.14 |
| NaCl | | | | 0.42 |
| Glycerin | 1 | 1 | 1 | |
| Purogene ® | 0.37 | 0.37 | 0.37 | 0.37 |
| (2.15 w/v %) | | | | |
| pH | 7.31 | 7.38 | 7.37 | 7.39 |
| Viscosity (cps) | | | | |
| Osmolality (mOsm) | 285 | 288 | | 285 |
| Particle Mean Size (μm) | 0.125 | 0.136 | 0.16 | 0.1375 |
| 99% Cumulative Size | 0.248 | 0.291 | 0.31 | 0.253 |
| (μm) | | | | |

TABLE 10

| | Example 20 | Example 21 |
|---|---|---|
| Ingredient | % w/w | % w/w |
| PHMB (ppm) | 1.1 | 1.1 |
| HPMC | 0.15 | 0.15 |
| Propylene Glycol | 0.5 | 0.5 |
| Dibasic Sodium Phosphate 7H₂0 | 0.12 | 0.12 |
| Monobasic Phosphate H₂0 | 0.01 | 0.01 |
| EDTA | 0.01 | 0.01 |
| NaCl | 0.55 | 0.55 |
| KCI | 0.14 | 0.14 |
| Vitamin E Acetate | 1.25 | 1.25 |
| Lumulse GR-40 | 0.5 | |
| TPGS | | 1 |

TABLE 11

| | Example 22 | Example 23 | Example 24 |
|---|---|---|---|
| Ingredient | % w/w | % w/w | % w/w |
| GRH-40 | 1 | 1 | 1 |
| Castor Oil | 1.25 | 1.25 | 1.25 |
| Cyclosporin A | 0.10 | 0.10 | |
| Brimonidine* tartrate | | | 0.15 |
| Boric Acid | 0.6 | 0.6 | 0.6 |
| Sodium Borate 10H₂0 | 0.035 | 0.035 | 0.035 |
| CaCl₂.2H₂O | 0.006 | 0.006 | 0.006 |
| MgCl₂.6H₂O | 0.006 | 0.006 | 0.006 |
| KCl | 0.14 | 0.14 | 0.14 |
| NaCl | 0.25 | 0.25 | 0.25 |
| Carboxymethylcellulose | | 0.50 | |
| Purogene ® (2.15 w/v %) | 0.35 | | 0.23 |
| pH | 7.4 | 7.4 | 7.2 |

*Brimonidine = (5-bromo-N-(4,5-dihydro-1H-imidazol-2-yl)-6-quinoxalinamine)

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A self-emulsifying composition comprising:
oil globules having an average size of less than 1 micron dispersed in an aqueous phase, said globules comprising:
(a) a surfactant component consisting essentially of one or two surfactants; and
(b) a polar oil component, said surfactant component and said oil component selected to self-emulsify when mixed without mechanical homogenization.

2. The self-emulsifying composition of claim 1, wherein the surfactant component has a hydrophobic portion which comprises a first part oriented proximal to the aqueous phase that is larger than a second part of the hydrophobic portion of the surfactant component oriented towards the interior of the oil globule.

3. The self-emulsifying composition of claim 2, wherein the surfactant component consists essentially of one surfactant with the first part of the hydrophobic portion of the surfactant that contains more atoms than the second part of the hydrophobic portion of the surfactant.

4. The self-emulsifying composition of claim 2, wherein the surfactant component consists essentially of two surfactants, a first of said surfactants comprising a first hydrophobic portion and a second of said surfactants comprising a second hydrophobic portion, said first hydrophobic portion having a longer chain length than the second hydrophobic portion.

5. A self-emulsifying composition according to claim 1, further comprising an additional surfactant that does not interfere with self-emulsification.

6. The self-emulsifying composition of claim 1, wherein the oil component comprises castor oil or a natural oil.

7. The self-emulsifying composition of claim 1, wherein the surfactant component is selected from the group consisting of a compound having at least one ether formed from at least about 1 to 100 ethylene oxide units and at least one fatty alcohol chain having from at least about 12 to 22 carbon atoms; a compound having at least one ester formed from at least about 1 to 100 ethylene oxide units and at least one fatty acid chain having from at least about 12 to 22 carbon atoms; a compound having at least one ether, ester or amide formed from at least about 1 to 100 ethylene oxide units and at least one vitamin or vitamin derivative; and combinations thereof consisting of no more than two surfactants.

8. The self-emulsifying composition of claim 1, wherein the surfactant component consists of one surfactant which is Lumulse GRH-40.

9. The self-emulsifying composition of claim 1, wherein the surfactant component consists of one surfactant which is TGPS.

10. The self-emulsifying composition of claim 1, wherein the oil globules have an average size of less than 0.25 micron.

11. The self-emulsifying composition of claim 1, wherein the oil globules have an average size of less than 0.15 micron.

12. A therapeutic composition comprising the self-emulsifying composition of claim 1 and a therapeutic drug.

13. An ophthalmic composition comprising the self-emulsifying composition of claim 1 and a drug that is therapeutic when administered to the eye.

14. The therapeutic composition of claim 12 which comprises a therapeutic compound selected from the group consisting of cyclosporin, prostaglandins, Brimonidine, and Brimonidine salts.

15. A method of preparing a self-emulsifying composition comprising:
preparing an oil phase comprising a polar oil and a surfactant component that consists essentially of one or two surfactants, wherein the polar oil and the surfactant component in the oil phase are in the liquid state;
preparing an aqueous phase at a temperature that permits self-emulsification; and
mixing the oil phase and the aqueous phase to form an emulsion, without mechanical homogenization.

16. A method of preparing a self-emulsifying composition according to claim 15, further comprising forming a paste between the oil phase and a part of the aqueous phase and mixing the paste with the rest of the aqueous phase to form an emulsion.

17. A self-emulsifying composition capable of being produced by the steps of:
preparing an oil phase comprising a polar oil and a surfactant component that consists essentially of one or two surfactants, wherein the polar oil and the surfactant component in the oil phase are in the liquid state;
preparing an aqueous phase at a temperature that permits self-emulsification; and
mixing the oil phase and the aqueous phase to form an emulsion, without mechanical homogenization.

18. The self-emulsifying composition of claim 17, wherein the surfactant component consists of a single surfactant.

19. The self-emulsifying composition of claim 17, wherein the oil phase comprises either a natural oil or castor oil.

20. The self-emulsifying composition of claim 17, wherein the surfactant component is selected from the group consisting of a compound having at least one ether formed from at least about 1 to 100 ethylene oxide units and at least one fatty alcohol chain having from at least about 12 to 22 carbon atoms; a compound having at least one ester formed from at least about 1 to 100 ethylene oxide units and at least one fatty acid chain having from at least about 12 to 22 carbon atoms; a compound having at least one ether, ester or amide formed from at least about 1 to 100 ethylene oxide units and at least one vitamin or vitamin derivative; and combinations thereof consisting of no more than two surfactants.

21. The self-emulsifying composition of claim 17, wherein the surfactant component consists of one surfactant which is Lumulse GRH-40.

22. The self-emulsifying composition of claim 17, wherein the surfactant component consists of one surfactant which is TGPS.

23. A therapeutic composition comprising the self-emulsifying composition of claim 17 and a therapeutic drug.

24. An ophthalmic composition comprising the self-emulsifying composition of claim 17 and a drug that is therapeutic when administered to the eye.

25. The therapeutic composition of claim 23 which comprises a therapeutic compound selected from the group consisting of cyclosporin, prostaglandins, Brimonidine, and Brimonidine salts.

26. A therapeutic composition comprising the self-emulsifying composition of claim 8 and a therapeutic drug.

27. An ophthalmic composition comprising the self-emulsifying composition of claim 8 and a drug that is therapeutic when administered to the eye.

28. The therapeutic composition of claim 26 which comprises a therapeutic compound selected from the group consisting of cyclosporin, prostaglandins, Brimonidine, and Brimonidine salts.

29. A therapeutic composition comprising the self-emulsifying composition of claim 21 and a therapeutic drug.

30. An ophthalmic composition comprising the self-emulsifying composition of claim 21 and a drug that is therapeutic when administered to the eye.

31. The therapeutic composition of claim 29 which comprises a therapeutic compound selected from the group consisting of cyclosporin, prostaglandins, Brimonidine, and Brimonidine salts.

32. A therapeutic composition comprising the self-emulsifying composition of claim 1 and a therapeutic drug, wherein the polar oil is either castor oil or a natural soil.

33. A therapeutic composition comprising the self-emulsifying composition of claim 17 and a therapeutic drug, wherein the polar oil is either castor oil or a natural oil.
